(19) United States
(12) Patent Application Publication
(10) Pub. No.: US 2022/0289809 A1
(43) Pub. Date: Sep. 15, 2022

(54) USE OF RELAXIN TO TREAT ATRIAL FIBRILLATION

(71) Applicant: University of Pittsburgh - Of the Commonwealth System of Higher Education, Pittsburgh, PA (US)

(72) Inventors: David S. Schwartzman, Pittsburgh, PA (US); Sanjeev G. Shroff, Pittsburgh, PA (US); Guy Salama, Pittsburgh, PA (US); Charles F. McTiernan, Pittsburgh, PA (US)

(73) Assignee: University of Pittsburgh - Of the Commonwealth System of Higher Education, Pittsburgh, PA (US)

(21) Appl. No.: 17/519,838

(22) Filed: Nov. 5, 2021

Related U.S. Application Data
(63) Continuation of application No. 16/509,239, filed on Jul. 11, 2019, now abandoned.
Application No. 16/509,239 is a continuation of application No. 15/482,214, filed on Apr. 7, 2017, now abandoned.
Application No. 15/482,214 is a continuation of application No. 14/434,681, filed on Apr. 9, 2015, now abandoned.
(60) Provisional application No. 61/712,234, filed on Oct. 10, 2012.

(51) Int. Cl. C07K 14/575; A61K 45/06; A61K 38/22

(52) U.S. Cl. CPC C07K 14/575; A61K 45/06; A61K 38/2221

(57) ABSTRACT

Disclosed herein are methods of using relaxin polypeptides and analogs, or nucleic acid molecules encoding such polypeptides to treat or inhibit atrial fibrillation.

### CROSS REFERENCE TO RELATED APPLICATIONS

This is a continuation of U.S. application Ser. No. 16/509,239, filed on Jul. 11, 2019, which is a continuation of U.S. application Ser. No. 15/482,214, filed on Apr. 7, 2017, which is a continuation of U.S. application Ser. No. 14/434,681, filed on Apr. 9, 2015, which is the U.S. National Stage of International Application No. PCT/US2013/064388, filed Oct. 10, 2013, which was published in English under PCT Article 21(2), which in turn claims the benefit of U.S. Provisional Application No. 61/712,234, filed Oct. 10, 2012; each of the prior applications is incorporated herein in its entirety.

### ACKNOWLEDGMENT OF GOVERNMENT SUPPORT

This invention was made with government support under Grant No. RR024153 awarded by the National Institutes of Health. The government has certain rights in the invention.

### FIELD

This disclosure relates to therapeutic use of relaxin to inhibit or treat atrial fibrillation.

### BACKGROUND

Relaxin (RLX) is a peptide hormone of the insulin superfamily, processed from a Preproprotein to form to the mature hormone containing A and B peptide chains, connected by two interchain disulfide bridges, and one intrachain disulfide within the A chain. RLX has been studied in reproduction, in which it functions to inhibit uterine contraction and induce growth and softening of the cervix. RLX interacts with the RLX receptor LGR7 (RXFP1) and LGR8 (RXFP2), which belong to the G protein-coupled receptor superfamily.

Atrial fibrillation is the most common form of cardiac arrhythmia and contributes significantly to cardiac morbidity and mortality. Current treatments for atrial fibrillation involve the destruction of viable tissue or risk of cardiac toxicity. Accordingly, there is an urgent need for novel therapeutic methods for treating atrial fibrillation.

### SUMMARY

Disclosed herein is the surprising discovery that systemic administration of RLX reduces the occurrence of atrial fibrillation in an animal model. When administered for a two-week period, RLX had multi-fold affects, including reducing fibrosis and myocyte hypertrophy, and increasing sodium channel expression in cardiac myocytes. Together, these actions of RLX lead to a marked increase in conduction velocity, and a reduction in fibrillation events. These exciting new findings show that RLX acts in a complex multifaceted manner on the extracellular matrix through fibroblast modifications and directly at the myocyte level.

Based on the discoveries presented herein, methods of treating and/or inhibiting atrial fibrillation are provided. In some embodiments, the methods include administering systemically a therapeutically effective amount of relaxin to a subject with or at risk of atrial fibrillation, thereby inhibiting or treating atrial fibrillation in the subject.

In some embodiments, the method includes administration of relaxin-1, relaxin-2, or relaxin-3. Further, in some embodiments, the subject has one of first detected, paroxysmal, persistent, or chronic atrial fibrillation. In additional embodiments, the subject (such as a subject with one of first detected, paroxysmal, persistent, or chronic atrial fibrillation) is selected for treatment.

In some embodiments, the methods include administering from 0.1 to 0.5 mg/kg/day relaxin to the subject, such as administration of about 0.5 mg/kg/day relaxin to the subject. The relaxin can be administered for particular periods of time, such as from one to two weeks.

The foregoing and other features and advantages of this disclosure will become more apparent from the following detailed description of a several embodiments which proceeds with reference to the accompanying figures.

### FIGURES

FIGS. 1A-1F are a set of graphs illustrating the inducibility of atrial fibrillation (AF) in normotensive and hypertensive rats. A-B: Representative action potential (AP) traces from the left atrium (LA) in normotensive Wystar-Kyoto (WKY) rat. As expected a premature pulse with an S1-S2 interval of 50 ms is longer than the refractory period and captures (FIG. 1A)) whereas S1-S2=45 ms is shorter than the refractory period and does not capture (FIG. 1B). No arrhythmias could be elicited by programmed stimulation or burst pacing. C-F: Representative AP traces from LA of spontaneously hypertensive rats (SHR). With a premature pulse at S1-S2=75 ms, a normal AP captures and propagates (FIG. 1C); an impulse delivered at a shorter interval of 70 ms elicits transient arrhythmia (FIG. 1D) and when delivered at still shorter interval of 60 ms, sustained AF are induced (FIG. 1E) which persisted for the duration of the experiment (FIG. 1F).

FIGS. 2A-2D are a set of graphs illustrating analysis of AF. A: Activation pattern on a 100×100 pixel CMOS with spatial resolution of 150×150 μm² exhibiting a single reentrant circuit during non-sustained AF. B: Activation pattern illustrating the creation and annihilation of multiple daughter waves (wavebreaks) during sustained AF. C: Time-frequency analysis of AF. The spectrogram was generated for each pixel by calculating the FFT spectrum for a brief Gaussian window of 2 seconds then shifting the window step-wise in time (Δt=1 ms) and re-measuring the FFT spectrum at successive t intervals. Top, Optical trace; Left, Overall FFT spectra; Contour map, spectrogram with isolines drawn every 12.5% of maximum. Spectrogram plots frequency (ordinate) versus time (abscissa) and is shown for 14 seconds of AF; the darker the color, the higher the energy density at that frequency. D: Histogram represents the dominant frequencies during sustained AF in SHR rats in the LA and the RA.

FIGS. 3A-3D are a set of graphs illustrating the effect of RLX on AF inducibility in SHR. The figures show examples of voltage (Vₘ) traces and activation maps from the LA of SHR+RLX (A to D) and from SHR+V hearts (E to H). A: An example of a non-sustained AF initiated by a single premature pulse using a short delay, S1-S2=35 ms. B: In most SHR+RLX hearts, no arrhythmias were elicited by a premature impulse and at S1-S2=30 ms the premature impulse failed to capture (n=7/8). Activation maps from an SHR+RLX heart at 250 ms (C) and 90 ms (D) S1-S2 interval, note the rapid propagation of the premature impulse in panel D.

FIGS. 4A-4D are a set of graphs illustrating the restitution kinetics of Action Potential Duration (APD) and Conduction Velocity (CV). A and B: Restitution kinetics (RK) measured from the left atrium (LA) for Conduction Velocity (CV) and action potential durations at 90% recovery to baseline (APD₉₀), respectively. CV and APD₉₀ were measured as a function of S1-S2 interval. For APD₉₀: WKY vs. SHR, p=NS; SHR vs. SHR+V, p<0.01; and SHR vs. SHR+RLX, p<0.01. For CV: WKY vs. SHR, p<0.05; SHR vs. SHR+V, p=NS; SHR vs. SHR+RLX, p<0.01. C and D: Restitution kinetics from the right atrium (RA) for CV and APD₉₀ respectively. CV and APD were measured as a function of S1-S2 interval. APD₉₀: WKY vs. SHR, p<0.0; SHR vs. SHR+V, p<0.01; SHR vs. SHR+RLX, p<0.01. CV: WKY vs. SHR, p<0.01; SHR vs. SHR+V, p=NS; SHR vs. SHR+RLX, p<0.01. All values are reported as mean±SD.

FIGS. 5A and 5B are a set of graphs and digital images illustrating that fibrotic remodeling of atria and its reversal with RLX. There was no significant difference in collagen to tissue ratio in both the RA and LA between SHR and SHR+V. However, RLX treatment attenuated the fibrosis within 2 weeks since SHR+RLX had a significantly lower collagen/tissue ratio when compared to SHR and SHR+V (p<0.05). A: LA and RA collagen I expression relative to tissue area for WKY, SHR, SHR+V, and SHR+RLX. All values are reported as mean±SD. Sample size n=3-5 per group. * p<0.05 versus WKY; † p<0.05 versus SHR; ‡ p<0.05 versus SHR+V. B: Representative immuno-histological sections at 20× magnification of age-matched male LA of SHR+RLX and SHR+V.

FIGS. 6A-6E are a set of graphs illustrating that RLX treatment decreases expression of fibrosis-related transcripts. Fold expression of TGFβ, MMP-2, MMP-9, collagen I, and collagen III relative to that of WKY treated with vehicle (V) in RNA isolated from left atria (LA). RLX: RLX treated. Values are mean±(SD). Sample size n=4-5 per group. * p<0.05 versus WKY+V; † p<0.05 versus SHR+V; ‡ p<0.05 versus WKY+RLX.

FIGS. 7A and 7B are a set of digital images and a graph illustrating that RLX treatment of human iPS-CMs doubles I_{Na} Density. Cardiomyocytes differentiated from human inducible pluripotent stem cells (iPSC) were cultured for 48 hours with a vehicle or 0.1 μM RLX. A: A representative image of human Y1 iPS cell derived CMs immuno-stained with anti-cTNT (Thermal Fisher) and anti-α-actinin (abeam) antibodies and counterstained with DAPI. The anti-cTNT and anti-α-actinin stains show substantial overlap. B: Current-to-voltage (I-V) relationships were measured and normalized with respect to cell capacitance. I-V plots for control and RLX treated human iPS-CMs demonstrate a marked ˜2-fold upregulation of Na⁺ current density (n=18 for each group, p=0.0023).

FIGS. 8A-8B are a set of graphs illustrating the effects of RLX treatment on RLX in Blood Serum and AP characteristics. A: Blood serum concentrations of recombinant RLX were measured pre and post treatment in SHR implanted with mini-pumps containing either RLX or V. RLX was not detected in any of the rats unless treated with RLX. Histograms shown as mean [RLX]±SEM B: Illustration of AP recorded from the LA of WKY and SHR hearts without and with 2-weeks of RLX treatment.

FIGS. 9A and 9B are a set of graphs illustrating the role of Caᵢ in AF inducibility. Superposition of AP (dark grey) and CaᵢT (light grey) from LA of SHR during (A) S1-S2=60 ms; (B) during the initiation of sustained AF at S1-S2=55 ms.

FIGS. 10 and 11 are a set of images and graphs illustrating AF in hearts from RLX treated SHR rats. 10: In SHR+V hearts, a single premature impulse at S1-S2=50 ms elicits AF that is sustained and does not stop spontaneously (lower trace) Panels 1-9: Activation maps from an SHR+V heart from 9 consecutive beats labeled 1-9 in the trace to depict the last 3 normal beats and the transition beats to AF panels 4-6 and 3 beats during AF panels 7-9. Note that in untreated SHR hearts the first spontaneous beat propagates at a slower CV (panel 6) compared to SHR+RLX panel 8 in FIG. 2SB. Isochronal lines are 1 ms apart. 11: In SHR+RLX hearts, a single premature impulse at S1-S2=35 ms elicits a brief transient tachycardia that self-terminates after one extra beat. Panels 1-5 show the activation maps of paced beats and panel 6 shows the activation that is interrupted by a premature impulse panel 7. The last beat propagates rapidly and self-terminates Isochronal lines are 1 ms apart.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (˜4 kb), which was created on Nov. 5, 2021, which is incorporated by reference herein.

### DETAILED DESCRIPTION

RLX (RLX), a peptide hormone, is thought to have a wide range of biological actions including anti-inflammatory, anti-apoptotic, cardioprotective, vasodilatory, proangiogenic effects, and anti-fibrotic effects. RLX was first identified for its role in reproduction and pregnancy. It is thought to play a critical role in the hemodynamic adaptive and anti-fibrotic changes that occur during pregnancy. Male RLX gene-deficient mice developed age-related cardiac fibrosis, ventricular stiffening, and diastolic dysfunction suggesting its role as an important intrinsic regulator of collagen turnover (Du et al., Cardiovasc Res. 2003; 57:395-404). However, prior studies did not identify a difference in atrial fibrillation between RLX-treated animal models and controls (Lekgabe et al., Hypertension. 2005; 46:412-418). Disclosed herein is the unexpected finding that systemic administration of RLX is useful for the treatment of atrial fibrillation. Based on this, methods of treating or inhibiting atrial fibrillation in a subject are described.

### I. ABBREVIATIONS

APD action potential duration
Caᵢ Intracellular free Ca²⁺
CMs cardiomyocytes
CV conduction velocity
iPSCs inducible pluripotent stem cells
iPS-CMs CMs derived from iPSCs
MMP metalloproteinase
RK restitution kinetics
RLX relaxin
SHR spontaneously hypertensive rat
TFD time frequency domain
Vₘ Membrane potential
WKY Wystar-Kyoto Rat

### II. Summary of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710).

The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will control.

To facilitate review of the various embodiments of this disclosure, the following explanations of terms are provided: Administration: The introduction of a composition or agent into a subject by a chosen route.

Administration can be local or systemic. For example, if the chosen route is intravenous, the composition is administered by introducing the composition into a vein of the subject. In some examples a disclosed therapeutic peptide, or a nucleic acid encoding the peptide, is administered to a subject. The term also encompasses long-term administration, such as is accomplished using a continuous release pump. In some embodiments, a therapeutic agent (e.g., relaxin) is administered systemically to a subject, for example, by intravascular (e.g., intravenous or intra-arterial), subcutaneous administration, or dermal or transcutaneous administration. In some embodiments, administration is not pericardial administration.

Agent: Any substance or any combination of substances that is useful for achieving an end or result; for example, a substance or combination of substances useful for inhibiting atrial fibrillation in a subject. Agents include proteins, nucleic acid molecules, compounds, small molecules, organic compounds, inorganic compounds, or other molecules of interest. An agent can include a therapeutic agent (such as an anti-atrial fibrillation agent), a diagnostic agent or a pharmaceutical agent. In some embodiments, the agent is a polypeptide agent (such as a therapeutic peptide). The skilled artisan will understand that particular agents may be useful to achieve more than one result.

Amino acid substitution: The replacement of one amino acid in polypeptide with a different amino acid.

Analog, derivative or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization, and so forth. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound.

Anti-atrial fibrillation agent: A molecule that decreases or reduces atrial fibrillation. In some examples, a RLX polypeptide, such as mature RLX-1 or mature RLX-2, is used as an anti-atrial fibrosis agent to inhibit, reduced or decrease atrial fibrosis in a subject. Additional anti-atrial fibrosis agents include, but are not limited to, Angiotensin Converting Enzyme (ACE) inhibitor, Angiotensin Receptor Blockers (ARBs) or Pirfenidone.

Atrial fibrillation (AF): AF is the most common form of cardiac arrhythmia (irregular heartbeat) and contributes significantly to cardiac morbidity and mortality. It is often associated with palpitations, fainting, chest pain, or congestive heart failure. However, in some people atrial fibrillation is caused by otherwise idiopathic or benign conditions. Atrial fibrillation has been associated with fibrosis, aging, and hypertension. Pharmacological therapy targeted at the underlying fibrotic substrate has claimed to be a new frontier in the management of atrial fibrosis.

AF can be identified clinically when taking a pulse, and the presence of atrial fibrillation can be confirmed with an electrocardiogram (ECG or EKG) which demonstrates the absence of P waves together with an irregular ventricular rate.

In atrial fibrillation, the normal regular electrical impulses generated by the sinoatrial node are overwhelmed by disorganized electrical impulses usually originating in the roots of the pulmonary veins, leading to irregular conduction of impulses to the ventricles which generate the heartbeat. Atrial fibrillation may occur in episodes lasting from minutes to days (“paroxysmal”), or be chronic in nature.

A guideline system is established for classifying atrial fibrosis (see, e.g., Fuster V, Rydén L E, Cannom D S et al. (2006). “ACC/AHA/ESC 2006 Guidelines for the Management of Patients with Atrial Fibrillation: a report of the American College of Cardiology/American Heart Association Task Force on Practice Guidelines and the European Society of Cardiology Committee for Practice Guidelines (Writing Committee to Revise the 2001 Guidelines for the Management of Patients With Atrial Fibrillation): developed in collaboration with the European Heart Rhythm Association and the Heart Rhythm Society”. Circulation 114 (7): e257-354). All atrial fibrillation patients are initially in the category called first detected atrial fibrosis. These patients may or may not have had previous undetected episodes. If a first detected episode self-terminates in less than 7 days and then another episode begins later on, the case has moved into the category of paroxysmal atrial fibrosis. Although patients in this category have episodes lasting up to 7 days, in most cases of paroxysmal atrial fibrosis the episodes will self-terminate in less than 24 hours. If instead the episode lasts for more than 7 days, it is unlikely to self-terminate, and it is called persistent atrial fibrosis. In this case, the episode may be still terminated by cardioversion. If cardioversion is unsuccessful or it is not attempted, and the episode is ongoing for a long time (e.g. a year or more), the patient's atrial fibrosis is called chronic.

Contacting: Placement in direct physical association; includes both in solid and liquid form, which can take place either in vivo or in vitro. Contacting includes contact between one molecule and another molecule, for example the amino acid on the surface of one polypeptide, such as a therapeutic peptide, that contacts another polypeptide. Contacting can also include contacting a cell for example by placing a peptide in direct physical association with a cell.

Control: A reference standard. In some embodiments, the control is a sample obtained from a healthy patient. In other embodiments, the control is a tissue sample obtained from a patient diagnosed with atrial fibrillation. In still other embodiments, the control is a historical control or standard reference value or range of values (such as a previously tested control sample, such as a group of patients having atrial fibrillation with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Conservative variants: “Conservative” amino acid substitutions are those substitutions that do not substantially affect or decrease the function of a protein. For example, the specific binding of a protein, such as RLX, for another protein to which it specifically binds, such as the RLX receptor. For example, a peptide that specifically binds another protein can include up to on, up to two, up to three, up to four, or up to five conservative amino acid substitutions, or at most about 1, at most about 2, at most about 3 at most about 4, at most about 5, at most about 10, or at most about 15 conservative substitutions and specifically bind the protein. The term conservative variation also includes the use of a substituted amino acid in place of an unsubstituted parent amino acid.

Furthermore, one of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of a protein, such as specific binding to another protein. For instance, if an amino acid residue is essential for a function of the protein, even an otherwise conservative substitution may disrupt that activity. Thus, a conservative substitution does not alter the basic function of a protein of interest.

Degenerate variant: In the context of the present disclosure, a “degenerate variant” refers to a polynucleotide encoding a polypeptide (such as a therapeutic peptide) that includes a sequence that is degenerate as a result of the genetic code. There are 20 natural amino acids, most of which are specified by more than one codon. Therefore, all degenerate nucleotide sequences encoding a polypeptide are included as long as the amino acid sequence of the polypeptide encoded by the nucleotide sequence is unchanged.

Expression: Translation of a nucleic acid into a protein. Proteins can be expressed and remain intracellular, can become a component of the cell surface membrane, or be can secreted into the extracellular matrix or medium.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Inhibiting or treating a disease: Inhibiting the full development of a disease or condition, for example, in a subject who is at risk for a disease such as atrial fibrillation. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated/purified: An “isolated” or “purified” biological component (such as a nucleic acid, peptide or protein) has been substantially separated, produced apart from, or purified away from other biological components in the cell of the organism in which the component naturally occurs, that is, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides and proteins prepared by recombinant expression in a host cell as well as chemically synthesized nucleic acids or proteins. The term “isolated” or “purified” does not require absolute purity; rather, it is intended as a relative term. Thus, for example, an isolated biological component is one in which the biological component is more enriched than the biological component is in its natural environment within a cell. Preferably, a preparation is purified such that the biological component represents at least 50%, such as at least 68%, at least 90%, at least 95%, or greater of the total biological component content of the preparation.

Linker: A molecule (e.g., a peptide) or group of atoms positioned between a first moiety and a second moiety (e.g., a peptide and an effector molecule). The linkage can be either by chemical or recombinant means. In several embodiments, a linker is bifunctional, i.e., the linker includes a functional group at each end, wherein the functional groups are used to couple the linker to the two moieties. The two functional groups may be the same, i.e., a homobifunctional linker, or different, i.e., a heterobifunctional linker.

In some embodiments, a linker is an amino acid sequence that covalently links two polypeptides. By way of example, in a recombinant polypeptide comprising two polypeptides domains, linker sequences can be provided between them, such as a polypeptide comprising a therapeutic peptide-linker-therapeutic peptide. Linker sequences, which are generally between 2 and 25 amino acids in length, are well known in the art and include, but are not limited to, the glycine(4)-serine spacer (GGGGS×3) described by Chaudhary et al., Nature 339:394-397, 1989.

The terms “conjugating,” “joining,” “bonding,” “labeling” or “linking” refer to making two molecules into one contiguous molecule; for example, linking two polypeptides into one contiguous polypeptide, or covalently attaching an effector molecule or detectable marker radionuclide or other molecule to a polypeptide.

Nav1.5: A sodium ion channel protein that in humans is encoded by the SCN5A gene. The Nav1.5 protein encoded by the SCN5A gene is an integral membrane protein and tetrodotoxin-resistant voltage-gated sodium channel subunit. The encoded protein is found primarily in cardiac muscle and is responsible for the initial upstroke of the action potential in an electrocardiogram. Defects in this gene are known to cause arrhythmia syndromes. The person of ordinary skill in the art is familiar with Nav1.5 protein and the encoding SCN5A gene, and their functions see, e.g., Rook et al., Cardiovascular Res., 93:12-23, 2012). The sequence of the SCN5A gene is known, see, e.g., GENBANK™ Gene ID NO. 6331, incorporated by reference herein as present in GENBANK on Oct. 10, 2013.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, e.g., a “recombinant polypeptide.” A recombinant nucleic acid may serve a non-coding function (e.g., promoter, origin of replication, ribosome-binding site, etc.) as well.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pericardial administration: A type of local (not systemic) administration of an agent directly to the pericardial fluid. Typically, pericardial administration involves invasive penetration of the pericardium to gain access to the pericardial fluid. Methods of accomplishing pericardial administration are known to the person of ordinary skill in the art (see, e.g., Maisch et al., Eds. Interventional Pericardiology, Springer: Heidelberg, 2011).

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the disclosed antibodies.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: Any chain of amino acids, regardless of length or post-translational modification (such as glycosylation, sulfation or phosphorylation). “Polypeptide” applies to amino acid polymers to naturally occurring amino acid polymers and non-naturally occurring amino acid polymer as well as in which one or more amino acid residue is a non-natural amino acid, for example an artificial chemical mimetic of a corresponding naturally occurring amino acid. In one embodiment, the polypeptide is a therapeutic peptide. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used interchangeably herein to refer to a polymer of amino acid residues.

Polypeptide Modifications: The present disclosure includes mutant polypeptides, as well as synthetic embodiments. In addition, analogues (non-peptide organic molecules), derivatives (chemically functionalized polypeptide molecules obtained starting with the disclosed polypeptide sequences) and variants (homologs) of polypeptides can be utilized in the methods described herein. The polypeptides disclosed herein include a sequence of amino acids that can be either L- and/or D-amino acids, naturally occurring and otherwise.

Peptides can be modified by a variety of chemical techniques to produce derivatives having essentially the same activity as the unmodified polypeptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the polypeptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the polypeptide side chains can be converted to C₁-C₁₆ alkoxy or to a C₁-C₁₆ ester using well-recognized techniques. Phenyl and phenolic rings of the polypeptide side chains can be substituted with one or more halogen atoms, such as F, Cl, Br or I, or with C₁-C₁₆ alkyl, C₁-C₁₆ alkoxy, carboxylic acids and esters thereof, or amides of such carboxylic acids. Methylene groups of the polypeptide side chains can be extended to homologous C₂-C₄ alkylenes. Thiols can be protected with any one of a number of well-recognized protecting groups, such as acetamide groups. Those skilled in the art will also recognize methods for introducing cyclic structures into the polypeptides of this disclosure to select and provide conformational constraints to the structure that result in enhanced stability. For example, a C- or N-terminal cysteine can be added to the polypeptide, so that when oxidized the polypeptide will contain a disulfide bond, generating a cyclic polypeptide. Other polypeptide cyclizing methods include the formation of thioethers and carboxyl- and amino-terminal amides and esters.

RLX: RLX is a peptide hormone of the insulin superfamily (reviewed in Sherwood OD. RLX's Physiological Roles and Other Diverse Actions. Endocr Rev. 2004; 25:205-34). Like insulin, RLX is 6 kilodalton protein processed from a prepro-form to the mature hormone containing A and B peptide chains, connected by two interchain disulfide bridges, and one intrachain disulfide within the A chain. Despite their structural similarity, RLX and insulin bind to distinct and unrelated receptors, and hence have no common cellular effects. The historical role of RLX has been in reproduction, in which it functions to inhibit uterine contraction and induce growth and softening of the cervix.

The RLX-like peptide family belongs in the insulin superfamily and consists of 7 peptides of high structural but low sequence similarity; RLX-1 (RLN1), 2 (RLN2) and 3 (RLN3), and the insulin-like (INSL) peptides, INSL3, INSL4, INSL5 and INSL6. The functions of RLX-3, INSL4, INSL5, INSL6 remain uncharacterized.

RLX protein and nucleic acid sequences are known, see for example, GENBANK Accession Nos. CAA00599.1, CAA00658.1, AA126416.1, AA126420.1, AAH05956.1, CAC04179.1, CAC04177.1, EAW84388.1, AAI40936.1, AAL40345.1, each of which is incorporated by reference herein as present in the data base on Oct. 10, 2012. The person of ordinary skill in the art will understand that RLX includes an A and B peptide chain and that a sequence including both of these chains can be processed into the respective chains to form mature RLX. Further, mature RLX can be purchased commercially, for example from Novartis AG, which purchased RLX from Corthera, Inc. (also known as RLX030, which is a recombinant form of human RLX.

RLX interacts with the RLX receptor LGR7 (RXFP1) and LGR8 (RXFP2), which belong to the G protein-coupled receptor superfamily. They contain a heptahelical transmembrane domain and a large glycosylated ectodomain, distantly related to the receptors for the glycoproteohormones. RLX receptors have been found in the heart, smooth muscle, the connective tissue, and central and autonomous nervous system. RLX receptor protein and nucleic acid sequences are known, see, for example, GENBANK Accession Nos. NP_001240662.1, NP_001240659.1, NP_001240657.1, NP_067647.2, NP_067647.2, NP_001240661.1, NP_570718.1, NP_001159530.1, NP_001159530.1, each of which is incorporated by reference herein as present in the data base on Oct. 10, 2012.

Sample (or biological sample): A biological specimen containing genomic DNA, RNA (including mRNA), protein, or combinations thereof, obtained from a subject. Examples include, but are not limited to, peripheral blood, tissue, cells, urine, saliva, tissue biopsy, fine needle aspirate, surgical specimen, and autopsy material.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1968; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

For sequence comparison of nucleic acid sequences and amino acids sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1968, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see for example, Current Protocols in Molecular Biology (Ausubel et al., eds 1995 supplement)). The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (World Wide Web address ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

Another indicia of sequence similarity between two nucleic acids is the ability to hybridize the sequences to each other, or to the same target sequence. The more similar are the sequences of the two nucleic acids, the more stringent the conditions at which they will hybridize. The stringency of hybridization conditions are sequence-dependent and are different under different environmental parameters. Thus, hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning: A Laboratory Manual, Cold Spring Harbor Laboratory Press, Cold Spring Harbor, N.Y., 2001; Tijssen, Hybridization With Nucleic Acid Probes, Part I: Theory and Nucleic Acid Preparation, Laboratory Techniques in Biochemistry and Molecular Biology, Elsevier Science Ltd., NY, NY, 1993; and Ausubel et al. Short Protocols in Molecular Biology, 4^{th} ed., John Wiley & Sons, Inc., 1999.

“Stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize. In contrast nucleic acids that hybridize under “low stringency conditions include those with much less sequence identity, or with sequence identity over only short subsequences of the nucleic acid.

Subject: Living multi-cellular vertebrate organisms, a category that includes human and non-human mammals. In one example, a subject is a human. In an additional example, a subject is selected that is in need of treatment and/or inhibition of atrial fibrillation. For example, the subject is either at risk of or has atrial fibrillation.

Therapeutically Effective Amount: An amount of a composition that alone, or together with an additional therapeutic agent(s) induces the desired response (e.g., inhibition or treatment of atrial fibrillation). In several embodiments, a therapeutically effective amount is the amount necessary to inhibit a sign or symptom of atrial fibrillation, and/or to inhibit atrial fibrillation in a subject, such as inhibiting the progression of atrial fibrillation in a subject. When administered to a subject, a dosage will generally be used that will achieve target tissue concentrations that has been shown to achieve a desired in vitro effect.

In one example, a desired response is to inhibit atrial fibrillation in a subject to which the therapy is administered. atrial fibrillation does not need to be completely eliminated for the composition to be effective. For example, a composition can decrease atrial fibrillation by a desired amount, for example by at least 10%, at least 20%, at least 50%, at least 60%, at least 70%, at least 80%, at least 90%, at least 95%, at least 98%, or even at least 100% (elimination of atrial fibrillation), as compared to a control, such as atrial fibrillation in the absence of the composition. In another example, a composition can decrease the progression of atrial fibrillation by a desired amount, for example by at least 10%, at least 20%, at least 50%, at least 60%, at least 70%, at least 80%, at least 90%, at least 95%, at least 98%, or even at least 100% (elimination of atrial fibrillation), as compared to a control, such as atrial fibrillation in the absence of the composition or to.

A therapeutically effective amount of an agent can be administered in a single dose, or in several doses, for example daily, during a course of treatment. However, the therapeutically effective amount can depend on the subject being treated, the severity and type of the condition being treated, and the manner of administration.

### III. Description of Several Embodiments

### A. Relaxin

In several embodiments, the disclosed methods include administration of a therapeutically effective amount of RLX, or a variant or analog of RLX, to a subject in need thereof, for example a subject with, or at risk of, atrial fibrillation. RLX (“RLX”) is a naturally occurring peptide hormone that plays an important physiological role within the body to orchestrate many of the maternal physiological responses to pregnancy. It is well established that RLX's ability to regulate collagen turnover is essential for softening the pelvic ligaments and female reproductive organs in preparation for child birth (Hisaw, 1926, Pro Soc Exp Bio. Med; 23:661-663; Schwabe et al., 1977, Biochem Biophys Res Comm; 75:503-570; James et al., 1977, Nature; 267:544-546). In addition to acting on the female reproductive system, RLX also affects non-reproductive targets, including the cardiovascular system and the connective tissue.

RLX is a member of a peptide hormone family that diverged from insulin early in vertebrate evolution and has been assigned to a specific hormone family, termed the RLX peptide family. The RLX peptide family includes three different RLXs, RLX-1, RLX-2 and RLX-3, as well as insulin-like peptide (INSL)3, INSL4, INSL5 and INSL6. All share high structural similarity with insulin due to the presence of six cysteine residues, which confer two inter-chain and one intra-chain disulfide bonds. Three RLX genes are present in humans. RLX-1 is found only in humans and the great apes and its expression is limited to the decidua, placenta and prostate. RLX-2 is the major circulating form of RLX in the human and the functional equivalent to the RLX-1 in all non-primates. RLX-3 has only recently been discovered and shows brain specific expression. Circulating RLX accounts for most of the known biological effects of the hormone in humans and experimental animals. See, for example, Bath, 2008, Vasc Health Risk Manag; 4(3):515-524. As used herein, RLX includes RLX-2 found in humans and great apes. In some embodiments of the present invention, RLX includes RLX-1, RLX-2 and/or RLX 3.

Like insulin, the structure of RLX is formed by the cleavage of a pro-hormone peptide into three chains (A, B and C), the removal of the C chain and the formation of three disulfide bridges between six invariant cysteine residues found on the A and B chains, to produce an active protein. Structurally, RLX is composed of A and B chains stabilized by inter- and intra-domain disulfide bonds with a molecular weight of approximately 6000 daltons. RLX for use in the disclosed embodiments includes, but is not limited to, RLX of a variety of species, including, but not limited to, porcine, murine, equine, shark, tiger, rat, dogfish, and human RLX.

The complete amino acid sequences and DNA sequences encoding the RLX polypeptide are known for a variety of species, including human RLX (see, for example, Hudson et al., 1983, Nature; 301, 628-631; Hayes, 2004, Reprod Biol Endocrinol; 2:36; Sherwood, 2004, Endocr Rev; 25(2):205-34; and Wilkinson et al., 2005, BMC Evolutionary Biology; 5:14). RLX protein and nucleic acid sequences are known, see for example, GENBANK Accession Nos. CAA00599.1, CAA00658.1, AAI26416.1, AAI26420.1, AAH05956.1, CAC04179.1, CAC04177.1, EAW84388.1, AAI40936.1, AAL40345.1, each of which is incorporated by reference herein as present in the data base on Oct. 10, 2012.

The person of ordinary skill in the art will understand that RLX includes an A and B peptide chain and that a Preproprotein sequence (such as those listed in the GenBank Accession nos. above) including both of these chains can be processed into the respective chains to form mature RLX. For example, in some embodiments, the RLX polypeptide includes an A chain and a B chain set forth as one of:

RLX-1: A Chain:
(SEQ ID NO: 1)
RPYVALFEKCCLIGCTKRSLAKYC,
B Chain:
(SEQ ID NO: 2)
KWKDDVIKLCGRELVRAQIAICGMSTWS
RLX-2: A Chain:
(SEQ ID NO: 3)
QLYSALANKCCHVGCTKRSLARFC,
B Chain:
(SEQ ID NO: 4)
DSWMEEVIKLCGRELVRAQIAICGMSTWS

Further, mature RLX can be purchased commercially, for example from Novartis AG, which purchased RLX from Corthera, Inc. (also known as RLX030, which is a recombinant form of human RLX). RLX includes RLX isolated from native sources and RLX produced using recombinant techniques, or chemically or enzymatically synthesized. In a preferred embodiment, RLX is human RLX, including, but not limited to, recombinant human RLX (“rhRLX”) (R&D Systems®, Minneapolis, Minn. and Corthera Inc., San Mateo, Calif.).

RLX analogs may be used in the methods and systems of the present invention. Such analogs may include, for example, the RLX analog B-R13/17K H2 (Hossain et al. “The chemically synthesized human RLX-2 analog, B-R13/17K H2, is an RXFP1 antagonist,” Amino Acids, 2010, 39: 409-416, incorporated by reference herein in its entirety) and cyclic and linear RLX peptide mimetics (Hossain et al., 2009 NY Acad Sci; 1160:16-19, incorporated by reference herein in its entirety). RLX variants may include, for example, RLX chimeras (Haugaard-Jönsson et al., 2009, NY Acad Sci; 1160:27-30, incorporated by reference herein in its entirety).

Through the use of recombinant DNA technology, RLX variants may be prepared by altering the underlying DNA. All such variations or alterations in the structure of the RLX molecule resulting in variants are included within the scope of this invention. Such variants include insertions, substitutions, or deletions of one or more amino acid residues, glycosylation variants, unglycosylated RLX, organic and inorganic salts, covalently modified derivatives of RLX, preproRLX, and proRLX. Such variant may maintain one or more of the functional, biological activities of the RLX polypeptide. Variants of RLX having such functional, biological activities can be readily identified using known in vitro or in vivo assays, such as any of those described in U.S. Pat. No. 5,945,402 and Lekgabe et al., 2005, Hypertension; 46:412-418. An anti-fibrotic agent of the present invention may be modified, for example, by PEGylation, to increase the half-life of the anti-fibrotic agent in the recipient, to retard clearance from the pericardial space, and/or to make the anti-fibrotic agent more stable for delivery by a pump.

In one embodiment, a RLX polypeptide useful within the disclosure is modified to produce peptide mimetics by replacement of one or more naturally occurring side chains of the 20 genetically encoded amino acids (or D-amino acids) with other side chains, for example with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from a 5-membered ring to a 4-, 6-, or 7-membered ring. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include furazanyl, furyl, imidazolidinyl, imidazolyl, imidazolinyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl groups. These heterocyclic groups can be substituted or unsubstituted. Where a group is substituted, the substituent can be alkyl, alkoxy, halogen, oxygen, or substituted or unsubstituted phenyl. Peptides, as well as peptide analogs and mimetics, can also be covalently bound to one or more of a variety of nonproteinaceous polymers, for example, polyethylene glycol, polypropylene glycol, or polyoxyalkenes, as described in U.S. Pat. Nos. 4,640,835; 4,496,668; 4,301,144; 4,668,417; 4,791,192; and 4,179,337.

In addition to the naturally occurring genetically encoded amino acids, amino acid residues in a RLX polypeptide may be substituted with naturally occurring non-encoded amino acids and synthetic amino acids. Certain commonly encountered amino acids which provide useful substitutions include, but are not limited to, β-alanine and other omega-amino acids, such as 3-aminopropionic acid, 2,3-diaminopropionic acid, 4-aminobutyric acid and the like; α-aminoisobutyric acid; ε-aminohexanoic acid; δ-aminovaleric acid; N-methylglycine or sarcosine; ornithine; citrulline; t-butylalanine; t-butylglycine; N-methylisoleucine; phenylglycine; cyclohexylalanine; norleucine; naphthylalanine; 4-chlorophenylalanine; 2-fluorophenylalanine; 3-fluorophenylalanine; 4-fluorophenylalanine; penicillamine; 1,2,3,4-tetrahydroisoquinoline-3-carboxylic acid; β-2-thienylalanine; methionine sulfoxide; homoarginine; N-acetyl lysine; 2,4-diaminobutyric acid; 2,3-diaminobutyric acid; p-aminophenylalanine; N-methyl valine; homocysteine; homophenylalanine; homoserine; hydroxyproline; homoproline; N-methylated amino acids; and peptoids (N-substituted glycines).

While in certain embodiments, the amino acids of a RLX polypeptide will be substituted with L-amino acids, the substitutions are not limited to L-amino acids. Thus, also encompassed by the present disclosure are modified forms of the SAHPs, wherein an L-amino acid is replaced with an identical D-amino acid (e.g., L-Arg→D-Arg) or with a conservatively-substituted D-amino acid (e.g., L-Arg→D-Lys), and vice versa.

Other peptide analogs and mimetics within the scope of the disclosure include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues (e.g., lysine or arginine). Acyl groups are selected from the group of alkyl-moieties including C3 to C18 normal alkyl, thereby forming alkanoyl aroyl species. Also embraced are versions of a native primary amino acid sequence which have other minor modifications, including phosphorylated amino acid residues, for example, phosphotyrosine, phosphoserine, or phosphothreonine, or other moieties, including ribosyl groups or cross-linking reagents.

In another embodiment, an additional functional domain or peptide can be linked to a RLX polypeptide or analog disclosed herein, creating a peptide/peptide analog-additional functional domain/peptide conjugate. The additional functional domain or peptide can be linked to the RLX polypeptide or peptide analog at either the N- and/or C-terminus.

Optionally, a linker can be included between the RLX polypeptide or analog and the additional functional domain or peptide. The linkers contemplated by the present disclosure can be any bifunctional molecule capable of covalently linking two peptides to one another. Thus, suitable linkers are bifunctional molecules in which the functional groups are capable of being covalently attached to the N- and/or C-terminus of a peptide. Functional groups suitable for attachment to the N- or C-terminus of peptides are well known in the art, as are suitable chemistries for effecting such covalent bond formation. The linker may be flexible, rigid or semi-rigid. Suitable linkers include, for example, amino acid residues such as Pro or Gly or peptide segments containing from about 2 to about 5, 10, 15, 20, or even more amino acids, bifunctional organic compounds such as H₂N(CH₂)ₙCOOH where n is an integer from 1 to 12, and the like. Examples of such linkers, as well as methods of making such linkers and peptides incorporating such linkers, are well-known in the art (see, e.g., Hunig et al., Chem. Ber. 100:3039-3044, 1974 and Basak et al., Bioconjug. Chem. 5:301-305, 1994).

Conjugation methods applicable to the present disclosure include, by way of non-limiting example, reductive amination, diazo coupling, thioether bond, disulfide bond, amidation and thiocarbamoyl chemistries. In one embodiment, the amphipathic alpha-helical domains are “activated” prior to conjugation. Activation provides the necessary chemical groups for the conjugation reaction to occur. In one specific, non-limiting example, the activation step includes derivatization with adipic acid dihydrazide. In another specific, non-limiting example, the activation step includes derivatization with the N-hydroxysuccinimide ester of 3-(2-pyridyl dithio)-propionic acid. In yet another specific, non-limiting example, the activation step includes derivatization with succinimidyl 3-(bromoacetamido) propionate. Further, non-limiting examples of derivatizing agents include succinimidylformylbenzoate and succinimidyllevulinate.

Also encompassed by the present disclosure are polypeptides including dimers, trimers, tetramers and even higher order polymers (i.e., “multimers”) comprising the same or different RLX polypeptide sequences. In multimers, the RLX polypeptide may be directly attached to one another or separated by one or more linkers. The RLX polypeptide can be connected in a head-to-tail fashion (i.e., N-terminus to C-terminus), a head-to-head fashion, (i.e., N-terminus to N-terminus), a tail-to-tail fashion (i.e., C-terminus to C-terminus), and/or combinations thereof. In one embodiment, the multimers are tandem repeats of two, three, four, and up to about ten RLX polypeptide, but any number of RLX polypeptide can be used.

### B. Polynucleotides and Expression

Nucleic acid molecules (also referred to as polynucleotides) encoding the RLX polypeptides provided herein can readily be produced by one of skill in the art. For example, these nucleic acids can be produced using the amino acid sequences provided herein, sequences available in the art, and the genetic code.

RLX polypeptides are provided above. One of skill in the art can readily use the genetic code to construct a variety of nucleic acid molecules encoding the RLX polypeptides, including functionally equivalent nucleic acids, such as nucleic acids which differ in sequence but which encode the same polypeptide, or encode a conjugate or fusion protein including the polypeptide and another protein.

Nucleic acid sequences encoding the disclosed RLX polypeptides can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence or by polymerization with a DNA polymerase using the single strand as a template. One of skill would recognize that while chemical synthesis of DNA is generally limited to sequences of about 500 bases, longer sequences may be obtained by the ligation of shorter sequences.

Exemplary nucleic acids can be prepared by cloning techniques. Examples of appropriate cloning and sequencing techniques, and instructions sufficient to direct persons of skill through many cloning exercises are found in Sambrook et al., supra, Berger and Kimmel (eds.), supra, and Ausubel, supra. Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), and Applied Biosystems (Foster City, Calif.), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

Any of the nucleic acids encoding any of the polypeptides disclosed herein (or fragment thereof) can be expressed in a recombinantly engineered cell such as bacteria, plant, yeast, insect and mammalian cells. In some embodiments, the polypeptides can be expressed as a fusion protein. The nucleic acid sequences can optionally encode a leader sequence.

It is expected that those of skill in the art are knowledgeable in the numerous expression systems available for expression of proteins including E. coli, other bacterial hosts, yeast, and various higher eukaryotic cells such as the COS, CHO, HeLa and myeloma cell lines.

One or more DNA sequences encoding the disclosed polypeptides can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

The expression of nucleic acids encoding the isolated proteins described herein can be achieved by operably linking the DNA or cDNA to a promoter (which is either constitutive or inducible), followed by incorporation into an expression cassette. The promoter can be any promoter of interest, such as a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signal for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, at the minimum, a strong promoter to direct transcription, a ribosome binding site for translational initiation (internal ribosomal binding sequences), and a transcription/translation terminator. For E. coli, this includes a promoter such as the T7, trp, lac, or lambda promoters, a ribosome binding site, and preferably a transcription termination signal. For eukaryotic cells, the control sequences can include a promoter and/or an enhancer derived from, for example, SV40 or cytomegalovirus, and a polyadenylation sequence, and can further include splice donor and/or acceptor sequences (for example, CMV splice acceptor and donor sequences). The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding the antibody, labeled antibody, or functional fragment thereof, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression systems such as plasmids and vectors of use in producing proteins in cells including higher eukaryotic cells such as the COS, CHO, HeLa and myeloma cell lines.

Modifications can be made to a nucleic acid encoding a polypeptide described herein without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps.

Once expressed, the polypeptides can be purified according to standard procedures of the art, including ammonium sulfate precipitation, affinity columns, column chromatography, and the like (see, generally, R. Scopes, PROTEIN PURIFICATION, Springer-Verlag, N.Y., 1982). The polypeptides need not be 100% pure. Once purified, partially or to homogeneity as desired, if to be used therapeutically, the polypeptides should be substantially free of endotoxin.

Methods for expression of polypeptides and/or refolding to an appropriate active form, from bacteria such as E. coli have been described and are well-known and are applicable to the antibodies disclosed herein. See, Buchner et al., Anal. Biochem. 205:263-270, 1992; Pluckthun, Biotechnology 9:545, 1991; Huse et al., Science 246:1275, 1989 and Ward et al., Nature 341:544, 1989.

Often, functional heterologous proteins from E. coli or other bacteria are isolated from inclusion bodies and require solubilization using strong denaturants, and subsequent refolding. During the solubilization step, as is well known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry 9: 5015-5021, 1970, and especially as described by Buchner et al., supra.

Renaturation is typically accomplished by dilution (for example, 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

A number of viral vectors have been constructed, that can be used to express the RLX polypeptides, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 66:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:66-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1166; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5368-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

### C. Compositions and Therapeutic Methods

The RLX polypeptides disclosed herein (including a plurality of such polypeptides), or polynucleotides encoding the RLX polypeptides (including a plurality of such nucleic acids), and vectors comprising the polynucleotides, can be used in methods of preventing, inhibiting and treating atrial fibrillation in a subject. In several embodiments, the methods can include selecting a subject in need of treatment, such as a subject with atrial fibrillation or at risk of developing atrial fibrillation. In some embodiments, the subject does not have congestive heart failure.

Any mode of administration can be used to provide the subject with the therapeutic compositions provided herein. Administration can be local or systemic. In several embodiments, a therapeutically effective amount of relaxin is administered to a subject, for example, by intravascular administration (such as intravenous or intraarterial administration), or subcutaneous administration. In some embodiments, the mode of administration is not pericardial administration. In other embodiments, the mode of administration is pericardial administration, for example, pericardial administration of a controlled release formulation including RLX. For example, the controlled release formulation can be a solid, semi-solid, or encapsulated liquid, which can be physically placed into the pericardial space. In some examples, the controlled release formulation is physically placed in the pericardial space by an instrument, such as a catheter or needle that is advanced transthoracically or intravascularly, or transmyocardially into the pericardial space.

In some embodiments, long-term administration is utilized, for example by using a continuous release pump. In further embodiments, prolonged administration is used, for example by administering a composition including relaxin in a controlled release formulation. Such modes of administration are known to the person of ordinary skill in the art and are further described herein.

In several embodiments, the RLX specifically binds to a RLX receptor on a cardiomyocyte in the subject. In some examples, the RLX receptor is RXFP1 or RXPF2.

The methods can be used either to avoid atrial fibrillation in a subject that does not have atrial fibrillation, or to treat existing atrial fibrillation in a subject. The subject with atrial fibrillation can have any stage of atrial fibrillation, such as first detected, paroxysmal, persistent or chronic atrial fibrillation. The person of ordinary skill in the art is familiar with methods of determining the category of atrial fibrillation in a subject (see, e.g., Fuster V, Rydén L E, Cannom D S et al. (2006). “ACC/AHA/ESC 2006 Guidelines for the Management of Patients with Atrial Fibrillation: a report of the American College of Cardiology/American Heart Association Task Force on Practice Guidelines and the European Society of Cardiology Committee for Practice Guidelines (Writing Committee to Revise the 2001 Guidelines for the Management of Patients With Atrial Fibrillation): developed in collaboration with the European Heart Rhythm Association and the Heart Rhythm Society”. Circulation 114 (7): e257-354). In several embodiments the methods include inhibiting the development of atrial fibrillation in a subject. Hence in some embodiments the methods involves selecting a subject at risk for atrial fibrillation, and administering RLX polypeptide, or a nucleic acid encoding the RLX, to the subject. Thus, the disclosed methods can be used to treat atrial fibrillation.

Treatment of atrial fibrillation can include delaying the development of atrial fibrillation in a subject, such as progression from persistent to chronic atrial fibrillation. Treatment of atrial fibrillation also includes reducing signs or symptoms associated with the atrial fibrillation. In some examples, treatment using the methods disclosed herein prolongs the time of survival of the subject.

Atrial fibrillation does not need to be completely eliminated for the methods to be effective. For example, treatment with one or more of the provided RLX polypeptides can decrease atrial fibrillation infection by a desired amount, for example by at least 10%, at least 20%, at least 50%, at least 60%, at least 70%, at least 80%, at least 90%, at least 95%, at least 98%, or even at least 100% (elimination of detectable atrial fibrillation), as compared to atrial fibrillation in the absence of the treatment. In additional examples, atrial fibrillation can be reduced or inhibited by the disclosed methods.

In some embodiments, administration of relaxin according to the disclosed methods results in a downgrade of the type of atrial fibrillation in a subject, for example, a downgrade from chronic to persistent atrial fibrillation, from chronic to paroxysmal atrial fibrillation, from chronic to first detected atrial fibrillation, from persistent to paroxysmal atrial fibrillation, from persistent to first detected atrial fibrillation, or from paroxysmal to first detected atrial fibrillation.

In further embodiments, administration of relaxin according to the disclosed methods delays progression of atrial fibrillation in a subject compared to a control, for example, the method can include a delay of progression from persistent to chronic atrial fibrillation, from persistent to chronic atrial fibrillation, from paroxysmal to chronic atrial fibrillation, from first detected to chronic atrial fibrillation, from paroxysmal to persistent atrial fibrillation, from first detected to persistent atrial fibrillation, or from first detected to paroxysmal atrial fibrillation.

In several embodiments, administration of a therapeutically effective amount of RLX to the subject reduces or inhibits atrial fibrosis in the subject.

For any application, treatment with the RLX polypeptide can be combined with additional therapy, such as treatment with another anti-atrial fibrillation agent, and/or an anti-fibrotic agent. For example the RLX polypeptides can be administered before, during or after administration of an anti-fibrotic agent, such as an Angiotensin converting enzyme (ACE) inhibitor, an Angiotensin Receptor Blocker (ARB), or a TGFβ inhibitor (e.g., Pirfenidone).

A therapeutically effective amount of the RLX polypeptide, or nucleic acid encoding the RLX polypeptide can be administered to a subject. A therapeutically effective amount of such molecules will depend upon the severity of the disease and/or infection and the general state of the patient's health. For example, a therapeutically effective amount of the RLX polypeptide is that which provides either subjective relief of a symptom(s) or an objectively identifiable improvement as noted by the clinician or other qualified observer.

In some embodiments, administering a therapeutically effective amount of relaxin includes administering about 1-1000 μg/kg/day RLX to a subject, such as about 1, 10, 50, 100, 150, 200, 250, 300, 350, 400, 450, 500, 550, 600, 650, 700, 750, 800, 850, 900, 950 or about 1000 μg/kg/day RLX to the subject. In one embodiment, the therapeutically effective amount includes administering about 500 μg/kg/day RLX to the subject. In some embodiments the therapeutically effective amount includes administering from 0.05 mg/kg/day to about 1.0 mg/kg/day relaxin to a subject in need thereof. In some embodiments, administering a therapeutically effective amount of RLX includes administering from 0.05 mg/kg/day to about 0.5 mg/kg/day relaxin to a subject in need thereof. In some embodiments the therapeutically effective amount includes administering from 0.03 mg/kg/day to about 0.3 mg/kg/day relaxin to a subject in need thereof. In some embodiments the therapeutically effective amount includes administering from 0.01 mg/kg/day to about 0.1 mg/kg/day relaxin to a subject in need thereof. In some embodiments the therapeutically effective amount includes administering from 0.1 mg/kg/day to about 0.5 mg/kg/day relaxin to a subject in need thereof.

In some embodiments, administering a therapeutically effective amount of RLX includes administering at least about 0.001 μg/kg/day RLX to the subject (such as at least about 0.0025 μg/kg/day, at least about 0.005 μg/kg/day, of at least about 0.01 μg/kg/day, at least about 0.025 μg/kg/day, at least about 0.05 μg/kg/day, at least about 0.1 μg/kg/day, at least about 0.25 μg/kg/day, at least about 0.5 μg/kg/day, at least about 1 μg/kg/day, at least about 2.5 μg/kg/day, at least about 5 μg/kg/day, at least about 10 μg/kg/day, at least about 25 μg/kg/day, at least about 50 μg/kg/day, at least about 100 μg/kg/day, at least about 250 μg/kg/day, at least about 0.5 mg/kg/day, at least about 1 mg/kg/day, at least about 5 mg/kg/day, at least about 10 mg/kg/day, at least about 25 mg/kg/day, at least about 50 mg/kg/day, at least about 100 mg/kg/day, of at least about 250 mg/kg/day, or at least about 500 mg/kg/day RLX to the subject). In some embodiments, administering a therapeutically effective amount of RLX includes administering at most about 0.001 μg/kg/day RLX to the subject (such as at most about 0.0025 μg/kg/day, at most about 0.005 jag/kg/day, at most about 0.01 μg/kg/day, at most about 0.025 μg/kg/day, at most about 0.05 μg/kg/day, at most about 0.1 μg/kg/day, at most about 0.25 μg/kg/day, at most about 0.5 μg/kg/day, at most about 1 μg/kg/day, at most about 2.5 μg/kg/day, at most about 5 μg/kg/day, at most about 10 μg/kg/day, at most about 25 μg/kg/day, at most about 50 μg/kg/day, at most about 100 μg/kg/day, at most about 250 μg/kg/day, at most about 500 μg/kg/day, at most about 1 mg/kg/day, at most about 25 mg/kg/day, at most about 5 mg/kg/day, at most about 10 mg/kg/day, at most about 25 mg/kg/day, at most about 50 mg/kg/day, at most about 100 mg/kg/day, at most about 250 mg/kg/day, or at most about 500 mg/kg/day RLX to the subject.

The disclosed methods can include a course of therapy, for example, in some embodiments, a subjected is administered relaxin for a period of time, such as at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 2.5 weeks, at least 3 weeks, at least 4 weeks, at least one month, at least two months, at least 3 months, at least 4 months, at least 5 months, at least 6 months, at least 7 months, at least 8 months, at least 9 months, at least 10 months, at least 11 months, at least one year, or more time. In one embodiment, the methods include administering from 0.05 mg/kg/day to about 1.0 mg/kg/day (such as about 0.05, about 0.1, or about 0.5 mg/kg/day) relaxin to the subject for at least one week, for about one week, for one week, for no more than one week, for one to two weeks, for two weeks, for no more than two weeks. In several embodiments, the subject is administered the therapeutically effective amount of the relaxin for a limited period of time, such as no more than one week, no more than two weeks, no more than three weeks, or nor more than four weeks.

Single or multiple administrations of the compositions including the RLX polypeptide, or nucleic acid encoding the RLX polypeptide, disclosed herein are administered depending on the dosage and frequency as required and tolerated by the patient. In any event, the composition should provide a sufficient quantity of at least one of the RLX polypeptides, or nucleic acid encoding the RLX polypeptide disclosed herein to effectively treat the patient. The dosage can be administered once but may be applied periodically until either a therapeutic result is achieved or until side effects warrant discontinuation of therapy. In one example, a dose of the RLX polypeptide is infused for thirty minutes every other day. In this example, about one to about ten doses can be administered, such as three or six doses can be administered every other day. In a further example, a continuous infusion is administered for about five to about ten days. The subject can be treated at regular intervals, such as monthly, until a desired therapeutic result is achieved. Generally, the dose is sufficient to treat or ameliorate symptoms or signs of disease without producing unacceptable toxicity to the patient.

In some embodiments, a subject is administered a first treatment with RLX, and a second treatment with RLX, with a “treatment holiday” between the two therapies. For example, a subject can be administered a first therapeutically effective amount of RLX (e.g., from 0.05 mg/kg/day to 0.5 mg/kg/day RLX for about two weeks) and then a second therapeutically effective amount of RLX (e.g., from 0.05 mg/kg/day to 0.5 mg/kg/day RLX for about two weeks) with a gap in treatment between administration of the first and second therapeutically effective amounts of RLX. The length of the gap in treatment can be determined by a treating physician based on the patient, and his or her reaction to the treatment. In some non-limiting examples, the gap in treatment is for about 1 week, about 2 week, about 3 weeks, about 4 weeks, about 5 weeks, about 6 weeks, about 7 weeks, about 8 weeks, about 3 months, about 4 months, about 5 months, about 6 months, about 7 months, about 8 months, about 10 months, about 11 months, about 12 months, or more time.

As disclosed herein, treatment with relaxin induces an increase in cardiac sodium channel current, and an increase in cardiac sodium channel expression in cardiac tissue. Accordingly, in several of the disclosed methods, administering a therapeutically effective amount of relaxin to a subject increases cardiac channel current and/or cardiac channel expression in the cardiac tissue of the subject. In several embodiments the increase in cardiac channel expression comprises an increase in NAv1.5 expression. The person of ordinary skill in the art is familiar with methods of measuring cardiac channel current and/or cardiac channel expression in a subject. For example, an increase in sodium current can be detected by monitoring the QRS complex of a subject (e.g., using an electrocardiogram) before and after treatment with the therapeutically effective amount of relaxin. The increase in sodium channel current can be detected by detecting a decrease in QRS duration in the subject before and after treatment using, for example, known methods, such as an electrocardiogram. Further, the expression level of cardiac sodium channels can be determined using known methods, e.g., by detecting expression level using an in vitro (e.g. on a biopsy sample) or in vivo antibody assay. In one non-limiting example, expression level is determined by detecting Nav1.5 protein level in a test sample compared to a control sample using an anti-Nav1.5 antibody. Nav1.5 antibodies are commercially available, for example, from Alamone labs, Cat. No. ASC-005.

Compositions are provided that include one or more of the RLX polypeptides, or nucleic acid encoding the RLX polypeptides, in a carrier. The compositions can be prepared with a pharmaceutically acceptable carrier, for example in unit dosage forms, for administration to a subject. The amount and timing of administration are at the discretion of the treating physician to achieve the desired purposes. The compositions can be formulated for systemic or local administration. In one example, the RLX polypeptide is formulated for parenteral administration, such as intravenous administration. In other examples, the pharmaceutical composition is formulated for intramuscular administration.

The compositions for administration can include a solution of the RLX polypeptide, or nucleic acid encoding the RLX polypeptide, in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of RLX polypeptide, or nucleic acid encoding the RLX polypeptide, in these formulations can vary widely, and will be selected primarily based on fluid volumes, viscosities, body weight and the like in accordance with the particular mode of administration selected and the subject's needs.

The composition including the RLX polypeptide, or nucleic acid encoding the RLX polypeptide can include additional agents, such as an anti-fibrotic agent, for example as described herein.

A typical pharmaceutical composition for intravenous administration includes about 0.1 to 10 mg of RLX polypeptide per subject per day. Dosages from 0.1 up to about 100 mg per subject per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed., Mack Publishing Company, Easton, Pa. (1995).

Expression vectors for RLX can be injected into the myocardium for sustained expression and delivery of RLX to the myocardium. The expression vectors can be targeted to specific myocardial areas, such as the sino-atrial node, or areas of known or suspected fibrosis in the heart that are thought to be involved in the initiation or propagation of fibrillation.

The RLX polypeptides, or nucleic acid encoding the RLX polypeptides, may be provided in lyophilized form and rehydrated with sterile water before administration, although they are also provided in sterile solutions of known concentration. The RLX polypeptide solution is then added to an infusion bag containing 0.9% sodium chloride, USP, and typically administered at a dosage of from 0.1 to 15 mg/kg of body weight. The person of ordinary skill in the art has considerable experience in administration of polypeptide therapeutics. Peptides can be administered by slow infusion, rather than in an intravenous push or bolus. In one example, a higher loading dose is administered, with subsequent, maintenance doses being administered at a lower level.

One approach to administration of nucleic acids is direct administration with plasmid DNA, such as with a mammalian expression plasmid. The nucleotide sequence encoding the disclosed RLX polypeptides can be placed under the control of a promoter to increase expression of the molecule. Administration of nucleic acid constructs is well known in the art and taught, for example, in U.S. Pat. Nos. 5,643,578, and 5,593,972 and 5,817,637. U.S. Pat. No. 5,880,103 describes several methods of delivery of nucleic acids encoding immunogenic polypeptides or other polypeptides to an organism. The methods include liposomal delivery of the nucleic acids.

In another approach for administering nucleic acids to a subject, the disclosed RLX polypeptides can also be expressed by attenuated viral hosts or vectors or bacterial vectors. Recombinant vaccinia virus, adeno-associated virus (AAV), herpes virus, retrovirus, cytomegalovirus, poxvirus or other viral vectors can be used to express the RLX polypeptide. For example, vaccinia vectors and methods of their use are described in U.S. Pat. No. 4,722,848.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Techonomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992) both of which are incorporated herein by reference.

Polymers can be used for ion-controlled release of the compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Techonomic Publishing Co., Inc., Lancaster, Pa. (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### EXAMPLES

The following examples are provided to illustrate certain particular features and/or embodiments. These examples should not be construed to limit the disclosure to the particular features or embodiments described.

### Example 1

RLX suppresses atrial fibrillation by reversing fibrosis and myocyte hypertrophy, and increasing conduction velocity and sodium current in spontaneously hypertensive rat hearts

### Abstract

Rationale: Atrial fibrillation (AF) contributes significantly to morbidity and mortality in elderly and hypertensive patients and has been correlated to enhanced atrial fibrosis. Despite a lack of direct evidence that fibrosis causes AF, reversal of fibrosis is considered as a plausible therapy.

Objective: To evaluate the efficacy of the anti-fibrotic hormone RLX at suppressing AF in spontaneously hypertensive rats (SHR).

Methods and Results: Normotensive Wistar Kyoto (WKY) and SHR were treated for 2-weeks with vehicle (WKY+V and SHR+V), or RLX (0.4 mg/kg/day, SHR+RLX) using implantable mini-pumps. Hearts were perfused, mapped optically to analyze action potential durations (APDs), intracellular Ca²⁺-transients, restitution kinetics (RK) and tested for AF vulnerability. SHR hearts had slower conduction velocity (CV) (p<0.01 vs. WKY), steeper CV RKs, greater collagen deposition, higher levels of transcripts for TGFβ, metalloproteinase-2, metalloproteinase-9, collagen I/III and reduced connexin-43 phosphorylation (p<0.05 vs. WKY). Programmed stimulation triggered sustained AF in SHR (n=5/5), SHR+V (n=4/4) but not in WKY (n=0/5) and SHR+RLX (n=1/8, p<0.01). RLX treatment reversed the transcripts for fibrosis, flattened CV-RK, reduced APD₉₀, increased CV (p<0.01) and reversed atrial hypertrophy (p<0.05). Independent of anti-fibrotic actions, RLX (0.1 μM) increased Na⁺-current density, I_{Na} (˜2-fold in 48-hours) in human cardiomyocytes derived from iPSCs (n=18/18, p<0.01).

Conclusions: RLX-treatment suppressed AF in SHR hearts by increasing CV from a combination of reversal of fibrosis and hypertrophy and increasing I_{Na}. The study provides compelling evidence that RLX may provide a novel therapy to manage AF in humans by reversing fibrosis, and hypertrophy and modulating cardiac ionic currents.

### INTRODUCTION

Atrial Fibrillation (AF), a disease associated with mortality, morbidity and high costs, affects tens of millions of people worldwide and is increasing in prevalence. Among the many risk factors that promote the development of AF, the most prominent are sex (more prevalent in males than females), old age (>60 years-old) and hypertension. Hypertension and aging lead to structural changes of the extracellular matrix (ECM) and enhanced AF vulnerability due to the altered myocardial substrate. Another etiology of AF is atrial tachycardia which leads to electrical remodeling and altered intracellular Ca²⁺ homeostasis associated with decreases in action potential duration (APD) and shortened atrial refractory periods. Fibrosis is a hallmark of arrhythmogenic ECM remodeling, occurs with alterations in connexin expression, and slows conduction velocity (CV), creating a barrier to impulse propagation by disrupting inter-myocyte coupling.

Increased collagen deposition has been well documented in AF patients compared with control subjects. Although the precise signaling processes of fibrosis are unknown multiple factors have been implicated (e.g. angiotensin II (All), Transforming Growth Factor β (TGF-β1) and Platelet Derived Growth Factor (PDGF)) in the pathogenesis of atrial fibrosis. ACE overexpression is associated with atrial enlargement, atrial fibrosis, and AF, whereas blockade of ACE blunts atrial fibrosis and AF in animal models and patients with HF. TGF-β1 and PDGF are thought to act on cardiac fibroblasts to increase collagen production without offsetting increases in collagen degradation.⁵ It should be noted that the role of fibrosis as the cause of AF can be overstated since some studies show no difference in fibrosis in AF and control patients. A possible explanation that remains unproven is that only some forms of collagen deposition cause AF; namely interstitial and/or disorganized collagen deposition promotes AF rather than surface collagen.

Current modalities for suppression of AF include drugs and ablation, each of which is limited by inefficacy, intolerance, and/or toxicity. Current drugs do not fundamentally alter the atrial substrate, whereas ablation requires destruction of viable tissue. Complications, costs, and difficulties associated with ablation have encouraged the development of better and safer drug therapies for the treatment of AF (Aliot, et al., Eur Heart J Suppl. 2008; 10:H32-H54; Chen, et al., Pace. 2003; 26:1301-1307). Existing anti-arrhythmic drug approaches have limited effectiveness and are associated with risks of serious complications, particularly ventricular pro-arrhythmia and/or organ toxicity (Fuster, et al., Circulation. 2006; 114:e257-354).

The Spontaneously Hypertensive Rat (SHR) has been widely studied as model of the effects of hypertension on the cardiovascular system (Okamoto, et al., Development of a strain of spontaneously hypertensive rats. Jpn Circ J. 1963; 27:282-293). In SHR, hypertension progresses as a function of age, is more pronounced in males than females, and exhibits most of the hallmarks of the human disease (Doggrell, et al., Cardiovascular Research. 1998; 39:89-105). Previous studies on the SHR model have shown an increased incidence of AF and atrial tachyarrhythmias compared to normotensive Wystar-Kyoto (WKY) rats, attributed to greater levels of fibrosis (Choisy, et al., Hypertension. 2007; 49:498-505). These findings suggest that fibrosis may promote the development of AF making it an important anti-arrhythmic target.

RLX (RLX), a pleiotropic hormone, which is widely conserved, has been shown to have a wide range of biological actions including anti-inflammatory, anti-apoptotic, cardioprotective, vasodilatory, pro-angiogenic effects, and anti-fibrotic effects (Bani, et al., Curr Drug Saf. 2009; 4:238-249; Conrad, et al., Curr Hypertens Rep. 2011; 13:409-420). RLX was first identified for its role in reproduction and pregnancy. It is thought to play a critical role in the hemodynamic adaptive and anti-fibrotic changes that occur during pregnancy (Conrad, et al., Curr Hypertens Rep. 2011; 13:409-420; Teichman, et al., Heart Fail Rev. 2009; 14:321-329; Conrad, K P, Semin Nephrol. 2011; 31:15-32). Male RLX gene-deficient mice developed age-related cardiac fibrosis, ventricular stiffening, and diastolic dysfunction suggesting an important role as an intrinsic regulator of collagen turnover (Du, et al., Cardiovasc Res. 2003; 57:395-404).

In the present report, it is demonstrated that exogenous systemic administration of RLX to spontaneously hypertensive rats suppresses AF inducibility by reversing fibrosis and hypertrophy, and increasing CV. These actions of RLX are relevant to human AF and as a proof-of-concept, it is shown that RLX upregulates I_{Na} in human iPS-CMs by a genomic mechanism.

### Methods

Study Design. All animals received humane care in a facility, in accordance with the “Guide for the Care and Use of Laboratory Animals” published by the NIH (publication 85-23, revised 1985). The study protocol was approved by the Institutional Animal Care and Use Committee of the University of Pittsburgh. AF inducibility was studied in age (9-12 months) and sex (male) matched rats (Charles River Laboratories) that were separated in four groups: 1) normotensive Wystar-Kyoto untreated rats (WKY); 2) untreated spontaneously hypertensive rats (SHR); 3) SHR treated with the vehicle saline (SHR+V); 4) SHR treated with RLX (SHR+RLX). Recombinant human RLX was supplied by Corthera-Novartis (Basel, CH). Osmotic mini-pumps (ALZET (Durect Corporation, model 2ML2) were used for the RLX and V treatment groups. Pumps were loaded with either recombinant human RLX solution (1.67 mg/ml) or V (20 mmol/L sodium acetate buffer, pH 5.0). The RLX infusion rate was ˜0.5 mg/kg/day (for 400 g rats) over the 14-day period. This dose of RLX is comparable to the dose previously used to treat in vivo rodent models of fibrosis (Lekgabe, et al., Hypertension. 2005; 46:412-418; Samuel, et al., Endocrinology. 2004; 145:4125-4133; Debrah, et al., J Appl Physiol. 2011; 111:260-271) and to examine RLX's effects on arterial hemodynamics and vascular mechanical properties (Debrah, et al., J Appl Physiol. 2011; 111:260-271; Debrah, et al., J Appl Physiol. 2005; 98:1013-1020). Pumps were surgically implanted under sterile technique into the subcutaneous space on the left side of anesthetized animals. Animals were monitored over the 14-days of RLX or V delivery to confirm proper healing of the implant pocket. Experiments showed that rats treated with the saline vehicle had as expected similar electrophysiological properties as untreated rats and as stated, data from the two groups were combined in some figures which also allowed us to display the findings more clearly. For western blot and RT-PCR analysis, the four groups were WKY treated with vehicle (WKY+V) or RLX (WKY+RLX), and SHR treated with vehicle (SHR+V) or RLX (SHR+RLX).

Physiological Measurements. Blood pressure, Heart Rate and Serum RLX Concentration were measured at 3 time-points of the treatment: pre (day 0), mid-(day 7) and post-treatment (day 14), as described in the supplement. Hearts were perfused in a Langendorff apparatus to simultaneously map action potentials (APs) and intracellular Ca²⁺ transients (CaTs) using standard techniques (see supplement)

Programmed Stimulation was used to test AF vulnerability, each heart was paced at the right atrium (RA) using a stimulation protocol consisting of 20 S1 pulses at 250 ms cycle length (CL) followed by a premature S2 pulse (see supplement). Maps of APs were used to calculate conduction velocity (CV), generate activation maps, measure APD₉₀ and investigate the nature of atrial fibrillation by time and frequency domain analysis using previously reported techniques. Transient AF lasted <3 s and self-terminated whereas sustained AF lasted >3 min and was terminated by a bolus injection of KCl (1M) in the compliance chamber located above the aortic cannula to the heart.

Tissue analysis. Atrial tissues were used to investigate changes in collagen deposition, connexin-43 phosphorylation, hypertrophy of cardiomyocytes and transcripts for fibrosis as described in the supplement. RT-PCR analysis was used to measure the expression levels of RNAs of interest which were normalized to GAPDH. Primer pair sequences (forward and reverse for each target, listed 5′ to 3′) used for RT-PCR are given in the supplement for: MMP-2, Collagen I, Collagen III, TGFβ and GAPDH.

Statistics. AF vulnerability between the different groups was compared using Fisher's exact test. Parameters recorded under different S1-S2 were compared using ANCOVA. For RT-PCR, western blot, and immune-fluorescence microscopy, comparisons among three or more groups were performed using a non-parametric test (Kruskal-Wallis) with post-hoc analyses (Conover). All results are reported as mean±SD unless otherwise stated. For all tests, a value of p<0.05 was considered to be statistically significant.

Blood pressure, Heart Rate and Serum RLX Concentration. Heart rate, diastolic and systolic blood pressures (BP) were measured using a tail cuff (Coda 6, Kent Scientific Corp., Torrington, Conn.), at 3 time-points of the treatment: pre (day 0), mid-(day 7) and post-treatment (day 14) (Marques et al., Hypertension. 2011; 57:477-483). Serum RLX concentration was assayed using a commercial kit (Quantikine Human RLX-2 Immunoassay, R&D Systems, Minneapolis, Minn., USA).

Optical Apparatus and Analysis. Rats were anesthetized with pentobarbital (50 mg/kg), injected with heparin (200 U/kg IV), then the heart was excised and perfused on a Langendorff apparatus with physiological Tyrode's solution containing (in mM): 122 NaCl, 25 NaHCO₃, 4.81 KCl, 2 CaCl₂, 2.75 MgSO₄, 5 Glucose (pH 7.4) gassed with 95 percent O₂ and 5 percent CO₂ at 37.0±0.2° C.

Hearts were placed in a chamber and perfused with blebbistatin (3-5 μM) for 5-10 min to arrest contractions and reduce motion artifacts; if needed blebbistatin perfusion was repeated ˜1-hour later. The hearts were stained with bolus injections of a voltage-sensitive dye (PGH-1; 300 μl of 1 mg/ml in dimethyl sulfoxide, DMSO) and a Ca²⁺ indicator (Rhod-2/AM, 300 μl of 1 mg/ml in DMSO), as previously described (Salama et al., Curr Protoc Cytom. 2009; Chapter 12:Unit 12 17).

Light from a 100-W tungsten-halogen lamp was collimated, passed through 530±30 nm interference filters, split by a 560 nm dichroic mirror and focused on the atria. Fluorescence from the stained heart was collected with tandem camera lenses (50 mm f/1.2 mm Nikon and 50 mm f/0.95 Navitar), was split with a 600 nm dichroic mirror to focus images of the atria at short (570-595 nm) and long (610-750 nm) wavelengths on two 100×100 pixel CMOS cameras (Ultima, Scimedia, Ltd. Tokyo, Japan). Each camera was scanned at 2,000 frames per second (Salama et al., Curr Protoc Cytom. 2009; Chapter 12:Unit 12 17). Pixel resolution was 150×150 μm (Salama et al., Curr Protoc Cytom. 2009; Chapter 12:Unit 12 17), and the data was recorded and stored in intervals of 4-8 seconds.

Activation and repolarization time points at each site were determined from fluorescence (F) signals by calculating (dF/dt)ₘₐₓ and (d²F/dt²)ₘₐₓ, which has been shown to coincide with ˜97% repolarization to baseline and recovery from refractoriness (Efimov et al., Circulation. 1994; 90:1469-1480). Action potential duration was measured from (dF/dt) max to 90% recovery to baseline, APD₉₀. Mean APD₉₀ was calculated for each heart by averaging APD₉₀ from a region of atrium consisting of 10×10 pixels or 100 APD₉₀ from each heart for a minimum of 5 hearts. Local conduction velocity (CV) vectors were calculated for each pixel from the differences in activation time-points of that pixel (determined from (dF/dt) max) and its 7×7 nearest neighbors, as previously described (Efimov et al., Circulation. 1994; 90:1469-1480). Local CVs were averaged and calculated as means±standard deviation (SD). Local CV can be overestimated when two wave fronts collide, transmural propagation breaks through the surface, or when activation appears synchronous over a region of the atrium because of its proximity to the pacing electrode. To avoid overestimations of CVs, CVs>1.25 m s⁻¹ were deleted from the analysis (Ziv et al., J Physiol-London. 2009; 587:4661-4680). Time and frequency domains analysis was achieved, as previously described (Choi et al., Circ Res. 2002; 91:339-345). APD Restitution Kinetics (RK) curves were generated by plotting mean APD₉₀ (from a minimum of 100 pixels per atrium times a minimum of 5 hearts (right or left atria)) versus S1-S2 interval in milliseconds. CV RK curves were generated by plotting the mean CV from a minimum of 5 atria vs. S1-S2 interval in milliseconds.

Programmed Stimulation. To test AF vulnerability, each heart was paced at the RA using a programmed stimulation protocol consisting of 20 S1 pulses at 250 ms cycle length (CL) followed by a premature S2 pulse with progressively shorter S1-S2 interval steps: 250 to 100 ms in 20 ms steps; 100 to 60 ms in 10 ms steps and 60 to 35 in 5 ms steps, until loss of capture or the initiation of AF.

Immunofluorescence Imaging. Atrial tissues were fixed in 2% paraformaldehyde, equilibrated in 30% sucrose, and flash frozen in supercooled isopentane. Frozen sections (7 micron thick) were cut by cryostat and sections interacted with rabbit anti-mouse collagen I (1:1000 dilution, Chemicon #AB765), Hoechst 33342 (1:1000 dilution, to identify nuclei, Sigma), and phalloidin 488 (1:250 dilution, Alexa Fluor 488 phalloidin A12379, Invitrogen, to identify filamentous actin). Fluorescent secondary antibodies included goat anti-rabbit IgG conjugated with Cy3 (1:1000 dilution, Molecular Probes #A10520, Invitrogen). To assess the severity of cardiac hypertrophy, left atrial cardiomyocytes were mounted on slides which were stained with Alexa-488-labelled wheat germ agglutinin (1:1000 dilution; Invitrogen, #W11261) to measure the cross sectional area of the cardiomyocytes, as previously described (McGaffin et al., Cardiovascular research. 2008; 77:54-63). For statistical significance, 10 myocytes were randomly selected from each of 5-10 sections of tissue from each group of rat hearts. Slides were viewed at 20× with a fluorescent microscope (Olympus Provis). Images from different wavelengths were collected with a cooled CCD camera at 24-bit gray depth and assembled (Adobe Photoshop). Collagen I to tissue area ratio was calculated as previously described using the area stained with phalloidin to index tissue area, averaging 6 random fields per heart, with 3-5 rats per group (Li et al., Proc Nat Acad Sci USA. 2000; 97:12746-12751), and analyzing right and left atria separately.

Analysis of connexin 43 phosphorylation. The relative phosphorylation status of connexin 43 in right atria of SHRs treated with a vehicle (n=4) or RLX (n=4) was measured using Western blots. Connexin 43 was focused on because previous reports failed to detect connexin 40 in rat atria (Gros et al., Bioessays. 1996; 18:719-730; Polontchouk et al., Journal of the American College of Cardiology. 2001; 38:883-891). Frozen right atria from SHR animals were homogenized in RIPA buffer (Thermo Cat #89900), containing protease (Cat #P8340 Sigma-Aldrich) and phosphatase inhibitors (Cat #5726 Sigma-Aldrich), and briefly centrifuged to remove gross debris. Protein levels were determined by Protein Assay (Bio-Rad) using bovine IgG as a standard. Proteins (25 μg/sample) were subjected to 12% polyacrylamide gel electrophoresis, transferred to PVDF membranes, interacted with antibody (Rabbit anti-Connexin 43, Invitrogen Cat #71-0700), washed, and developed by chemiluminescence. Digitized films were analyzed using NIH Image J to determine the ratio of the phosphorylated (43 kD) to non-phosphorylated (40 kD) connexin 43.

RT-PCR Analysis. RNA was isolated (RNAEasy, Qiagen) and copied to cDNA (High Capacity Reverse Transcription kit, Applied Biosystems) according to manufacturer protocols. A Syber-green-based formulation (Absolute Sybr-Green, Thermo Fischer Scientific, Waltham, Mass.) was utilized for fluorescence-based kinetic real-time PCR using an Applied Biosystems model 7000 detection system (Applied Biosystems Inc., Foster City, Calif.). Expression levels of RNAs of interest were normalized to that of GAPDH using the δδCt method (Livak et al., Methods. 2001; 25:402-408), and reported relative to the mean of the WTV group. Primer pair sequences (forward and reverse for each target, listed 5′ to 3′) used for RT-PCR are as follows; MMP-2: gcaccaccgaggattatgac (SEQ ID NO: 5), cacccacagtggacatagca (SEQ ID NO: 6); MMP-9: cctctgcatgaagacgacataa (SEQ ID NO: 7), ggtcaggtttagagccacga (SEQ ID NO: 8); Collagen I: catgttcagctttgtggacct (SEQ ID NO: 9), gcagctgacttcagggatgt (SEQ ID NO: 10); Collagen III: tcccctggaatctgtgaatc (SEQ ID NO: 11), tgagtcgaattggggagaat (SEQ ID NO: 12); TGFβ: cctggaaagggctcaacac (SEQ ID NO: 13), cagttcttctctgtggagctga (SEQ ID NO: 14); GAPDH: agctggtcatcaatgggaa (SEQ ID NO: 15), atttgatgttagcgggatc (SEQ ID NO: 16).

Cardiomyocyte (CM) differentiation of human iPS cells: iPS-CMs. A human Y1 iPS cell line was generated from human fibroblast line HDF-α as previously described (Lin et al., Cardiovasc Res. 2012; 95:327-335). The following conditions were used for cardiomyocyte differentiation (Yang et al., Nature. 2008; 453:524-528) using the basal StemPro®-34 (Invitrogen) medium as described in our previous study (Lin et al., Cardiovasc Res. 2012; 95:327-335): days 0-1, BMP4 (10 ng/ml); days 1-4, BMP4 (10 ng/ml), bFGF (5 ng/ml) and Activin A (1.5 ng/ml); days 4-8, DKK1 (150 ng/ml) and VEGF (10 ng/ml); after day 8, VEGF (10 ng/ml), bFGF (10 ng/ml), BMP4 (1 ng/ml) and DKK1 (150 ng/ml). Cultures were maintained in a 5% CO₂/5% O₂/90% N₂ environment for the first 20-days and were then transferred into a 5% CO₂/air environment. All cytokines were purchased from R&D Systems.

Cell Culture and RLX Treatment. Human iPS-CMs were seeded on 15 mm cover-slips coated with 0.01% (w/v) gelatin solution placed in 12-well plates. Cells are seeded at a density of 20,000-40,000 viable iPS-CMs per a dish in 2 mL of room temperature plating medium, permitting the cells to culture as single cells. Cells were incubated for at least 2 days at 37° C., 7% CO₂. Non-adherent cells are removed after 2 days by rinsing with basal differentiation medium. Plated iPS-CMs are maintained by changing the 2 mL of maintenance medium every 2 days. RLX treated coverslips contained 0.1 μM of Recombinant Human RLX (supplied by Corthera-Novartis (Basel, CH) for 48 hours prior to voltage-clamp experiments.

Voltage-Clamp Protocols. Cardiac action potentials and ionic currents were recorded from single iPSC derived myocytes. Ionic currents were recorded using the whole-cell patch clamp technique performed at room temperature using Axopatch1D, Digidata 1322A, and pClamp 9 (Axon Instruments) for data amplification, acquisition and analysis. Cells in Tyrode solution were kept in a recording chamber (300 μl volume) and were continuously perfused with fresh Tyrode solution. Suction pipettes, were fabricated from borosilicate glass using a Flaming/Brown horizontal micropipette puller with resistances between 2 and 4 MΩ. Action potentials were recorded in the current clamp mode and sodium current magnitudes were measured as the rapid peak inward current recorded in the same solution under voltage clamp mode. APs were elicited by a current injection through the patch, sufficient to elicit an upstroke. Patch pipettes contained the following intracellular solution (mM): 140 KCl, 1 MgCl₂, 5 EGTA, 5 ATP (Mg salt), 5 Na₂-creatinephosphate, 0.2 GTP, and 10 HEPES, pH 7.4 and extracellular solution contained (mM): 144 NaCl, 5.4 KCl, 1 MgCl, 2.5 CaCl₂, 5.6 glucose, and 10 HEPES, pH 7.4. Currents were elicited by a protocol of depolarizing potentials of −130 mV to 50 mV in 10 mV increments from a holding potential of −80 mV. Current densities were measured as the peak current for each potential pulse. Currents were normalized to the cell capacitance and expressed in pA/pF.

### Results

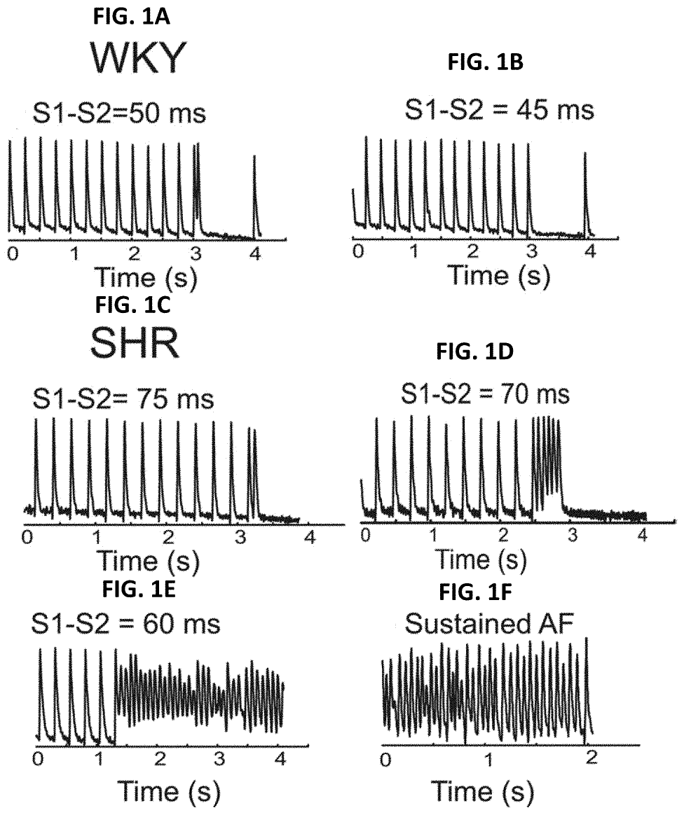

AF vulnerability. AF was inducible in each of 5 SHR animals, but none of 5 WKY animals (p<0.01, FIG. 1). In WKY hearts, a premature impulse close to the refractory period (S1-S2=50 ms) captured and propagated whereas still shorter intervals (S1-S2<50 ms) failed to capture and did not induce AF (n=0/5) (FIG. 1A-B). In SHR hearts (FIG. 1C-F), a premature impulse at S1-S2=75 ms, captured and propagated normally (C) but a 70 ms S1-S2 interval induced a transient arrhythmia (D) and a still shorter interval produced sustained AF (E and F) (n=5/5, p<0.01 vs WKY). In left atria while pacing at 250 ms CL, refractory periods (RP) were shorter than mean APD₉₀ (WKY: RP=40±13 ms, APD₉₀=98±18 ms, n=5, p<0.05; SHR: RP=58±10 ms, mean APD₉₀=87±18 ms, n=5, p<0.05). RPs were shorter in WKY vs. SHR atria (n=5 each, p<0.01) and in SHR hearts, sustained AF was initiated at S1-S2=70±12 ms which was not significantly different than their mean RP (n=5, p=NS).

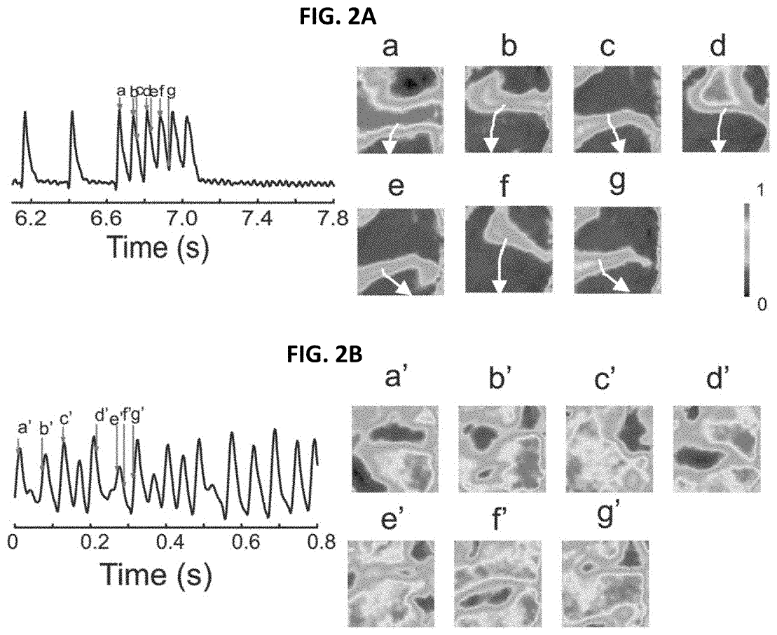

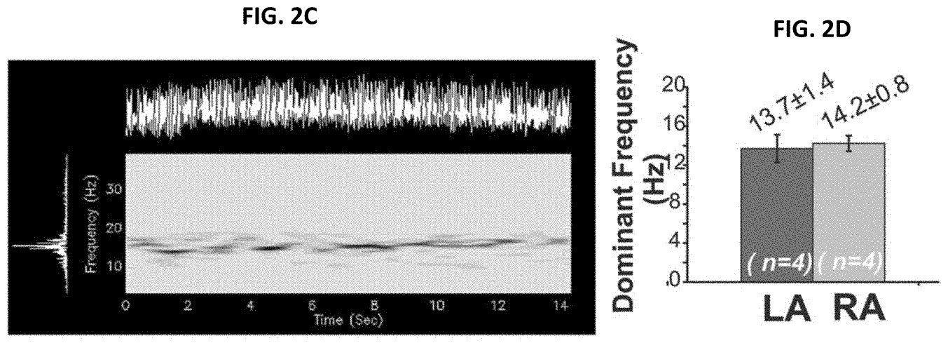

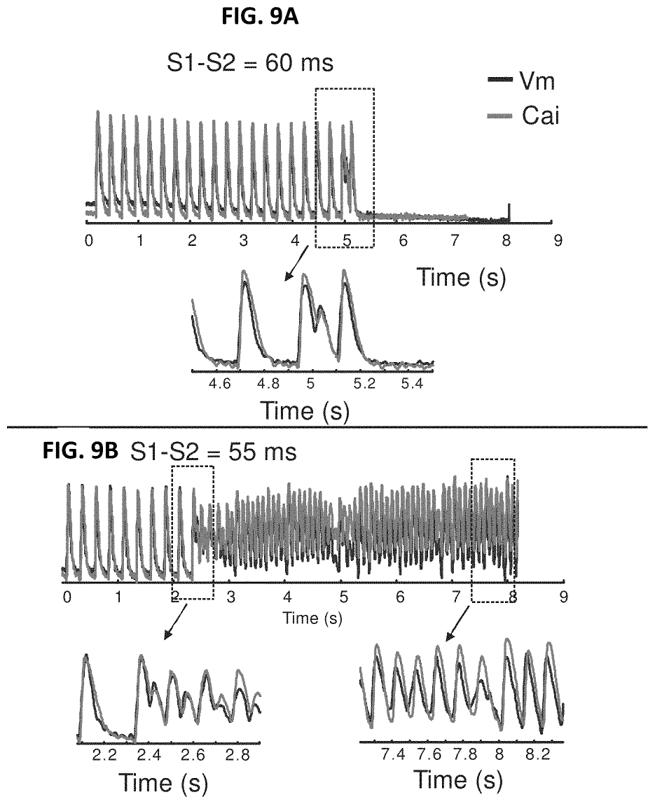

Optical Mapping of Atrial Fibrillation. FIG. 2 illustrates AP from an SHR heart before and during a transient AF (A) and during a sustained AF (B). Activation maps during transient (a-g) AF (A) exhibited a stable reentry pattern with wavefronts emanating from a similar origin and propagating in a similar direction from beat-to-beat. In contrast, during sustained AF (FIG. 2B: a′-g′), the origins of successive reentrant waves varied randomly and the arrhythmia was perpetuated by co-existing reentrant circuits maintained through the continuous annihilation and creation of daughter wavelets (Choi, et al., Circ Res. 2002; 91:339-345). Voltage oscillations during AF were analyzed in time and frequency domains to visualize the evolution of AF frequencies (Choi, et al., Circ Res. 2002; 91:339-345). The spectrogram (short-time Fourier transform) reveals co-existing reentrant circuits at different frequencies (9-20 Hz) and energy densities (FIG. 2C). The analysis showed that the right (RA) and left atria (LA) had similar dominant frequencies (13.7±1.4 and 14.2±0.8 Hz) (FIG. 2D). In SHR hearts, abnormalities in Ca²⁺ homeostasis (e.g. larger CaᵢTs and spark amplitudes, normal L-type Ca²⁺ current density, IC_{a,L} and absence of heart failure) has been attributed to cellular hypertrophy resulting in altered coupling between Ca²⁺-entry via IC_{a,L} and SR Ca²⁺-release (Shorofsky, et al., Circ Res. 1999; 84:424-434). The altered SR Ca²⁺ release in SHR hearts suggested a potential mechanism to initiate and/or sustain AF, which is tested by simultaneous mapping of APs and CaᵢT to search for spontaneous (non-voltage dependent) Ca²⁺-release and Caᵢ oscillations. As shown in FIG. 9A, Caᵢ followed Vₘ, during transient arrhythmia and sustained AF (FIG. 9B); neither did Caᵢ oscillations occur that were not associated by voltage depolarizations (n=4/4 hearts).

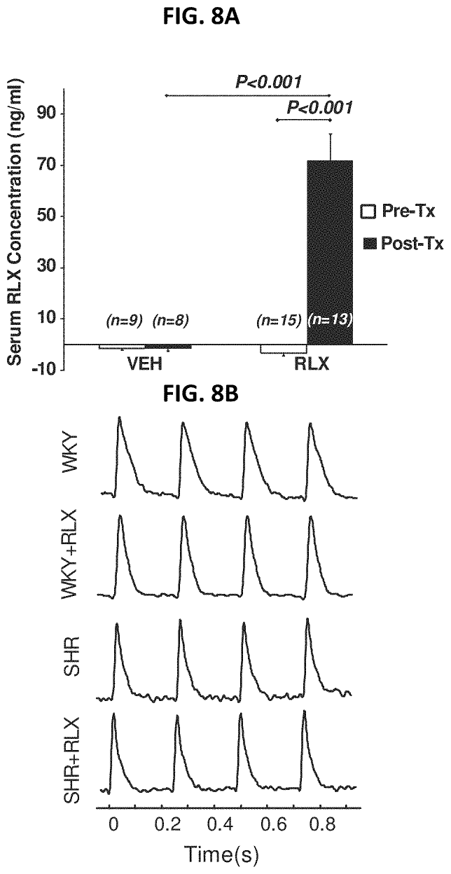

Effects of RLX treatment on blood pressure, heart rate, serum RLX and AP. RLX was not detectable in the serum of animals, unless administered exogenously. In SHR+RLX rats, serum RLX measured on the final day of treatment was 70±9 ng/ml whereas SHR+V rats had undetectable levels of RLX (p<0.001, see FIG. 8). Blood pressures were comparable between SHR+RLX and SHR+V animals at all-time points, indicating that RLX did not reverse the hypertension (see Table 2). RLX is known to cause an acute increase in heart rate, mediated by cAMP elevation consistent with the findings that RLX (100 nM) perfusion increased heart rate by 10-15% within a minute (n=5 per group: SHR or WKY). A similar increase in heart rate was found in SHRs in mid-treatment (1-week) and post-treatment (2-weeks) with RLX (Table 1).

TABLE 1
In Left Atria, effect of RLX on APD₉₀, CV and AP Rise-time vs. cycle length (CL)

| | APD₉₀ | | | | | | | |
|---|---|---|---|---|---|---|---|---|
| | WKY | SHR | SHR + V | SHR + RLX | CV | | | |
| CL | n = 5‡ | (n = 5) | (n = 4)* | (n = 5)* ‡ | WKY*‡ | SHR | SHR + V | SHR + RLX ‡ |
| 250 | 97 ± 16 | 93 ± 18 | 75 ± 6 | 76 ± 15 | 1.03 ± 0.2 | 0.93 ± .03 | 0.86 ± 0.2 | 1.17 ± 0.1 |
| 200 | 89 ± 14 | 90 ± 16 | 68 ± 8 | 82 ± 15 | 1.09 ± 0.2 | 0.92 ± 0.1 | 0.85 ± 0.1 | 1.15 ± .04 |
| 180 | 89 ± 14 | 88 ± 15 | 65 ± 6 | 76 ± 10 | 1.07 ± 0.2 | 0.91 ± 0.1 | 0.84 ± 0.1 | 1.09 ± 0.1 |
| 160 | 85 ± 12 | 85 ± 12 | 63 ± 5 | 71 ± 6 | 1.01 ± 0.1 | 0.94 ± 0.1 | 0.86 ± 0.1 | 1.15 ± 0.1 |
| 140 | 78 ± 6 | 81 ± 9 | 63 ± 5 | 62 ± 8 | 1.02 ± 0.1 | 0.92 ± 0.1 | 0.88 ± 0.2 | 1.20 ± 0.1 |
| 120 | 75 ± 1 | 75 ± 4 | 61 ± 6 | 63 ± 6 | 1.04 ± 0.1 | 0.90 ± 0.1 | 0.88 ± 0.2 | 1.15 ± 0.1 |
| 100 | 66 ± 3 | 67 ± 3 | 57 ± 4 | 60 ± 5 | 0.95 ± 0.2 | 0.78 ± 0.2 | 0.83 ± 0.1 | 1.02 ± 0.2 |
| 90 | 63 ± 1 | 63 ± 5 | 56 ± 2 | 55 ± 5 | 0.84 ± 0.1 | 0.70 ± 0.2 | 0.87 ± 0.1 | 1.02 ± 0.1 |

| | Rise Time | | | |
|---|---|---|---|---|
| CL | WKY | SHR | SHR + V | SHR + RLX |
| 250 | 30.5 ± 0.4 | 29.8 ± 1.3 | 29.1 ± 0.1 | 29.4 ± 0.4 |
| 200 | 30.6 ± 0.3 | 30.1 ± 1.5 | 29.2 ± 0.1 | 29.1 ± 0.2 |
| 180 | 30.4 ± 0.1 | 30.3 ± 1.6 | 29.1 ± 0.1 | 29.2 ± 0.1 |
| 160 | 30.8 ± 0.4 | 30.3 ± 1.7 | 29.0 ± 0.3 | 29.2 ± 0.2 |
| 140 | 30.7 ± 0.1 | 30.3 ± 1.8 | 29.2 ± 0.1 | 29.2 ± 0.2 |
| 120 | 30.6 ± 0.03 | 30.0 ± 1.5 | 29.2 ± 0.1 | 29.2 ± 0.1 |
| 100 | 30.2 ± 0.2 | 30.0 ± 2.3 | 29.4 ± 0.1 | 29.3 ± 0.4 |
| 90 | 29 ± 0.3 | 29.8 ± 2.4 | 29.2 ± 0.1 | 29.0 ± 0.1 |

For the data presented in Table 1, in each left atria, AP rise-time and APD₉₀ was measured from 10 pixels and averaged for 5 atria; CL, AP rise-time and APD₉₀ are in ms, CV in m/s, as means±SD. For WKY, n=5 hearts; SHR and SHR+RLX, n=5 hearts; and for SHR+V, n=4 hearts. *p<0.05 vs. SHR; ‡ p<0.05 vs. WKY, SHR and SHR+V, (ANCOVA).

TABLE 2
Effect of RLX treatment on blood pressure (BP) and heart rate (HR).

| | Pre-Tx | | Mid-Tx | | Post-Tx | |
|---|---|---|---|---|---|---|
| | SHR + RLX | SHR + V | SHR + RLX | SHR + V | SHR + RLX | SHR + V |
| Tail BP (mmHg) | 156 ± 20 (14) | 157 ± 34 (5) | 155 ± 24 (7) | 165 ± 12 (3) | 173 ± 14 (6)* | 164 ± 27 (4) |
| HR (BPM) | 427 ± 18 (10) | 393 ± 48 (5) | 465 ± 43 (7)* | 400 ± 78 (4) | 478 ± 27 (6)* | 430 ± 63 (4) |

For Table 2, Blood pressure (BP) and heart rate (HR) were measured before RLX treatment (Pre-Tx), midway or 1-week after RLX treatment (Mid-Tx) and after 2-weels of RLX treatment (Post-Tx). Mean values are given ±S.D, number of rats for each group is shown in parentheses; * versus Pre-TX (SHR+RLX) p<0.05

The effects of RLX or V treatment on APD₉₀, CV and AP rise-time (RT) on the left atria of SHR hearts were measured as a function of CL and compared to values measured in untreated SHR and WKY hearts. These electrical characteristics are shown for the left atria in Table 1, while the heart was paced on the right atria. APD₉₀ were shorter in SHR than WKY (p<0.05), shorter in and SHR+V than SHR (p<0.05) and shorter in SHR+LX than SHR+V (p<0.01) using ANCOVA. CV was slower in SHR and SHR+V than WKY hearts and SHR+RLX resulted in a marked increase in CV compared WKY, SHR and SHR+V (p<0.005, Table 1). AP rise-times tended to shorter in SHR than WKY hearts and SHR+RLX tended to further reduce rise times but these changes did not reach statistical significance. Similar results were obtained in the right atria while pacing on the RA, Table 3. The shape and time course of APs from WKY, WKY+RLX, SHR and SHR+RLX hearts are illustrated in FIG. 8B.

TABLE 3
In right Atria, effect of RLX on APD₉₀, CV and AP Rise-time of vs. cycle length (CL)

| | APD₉₀ | | | | CV | | | | Rise Time | | | |
|---|---|---|---|---|---|---|---|---|---|---|---|---|
| | WKY | SHR | SHR + V | SHR + RL | | | | SHR + | | | | SHR + |
| CL | (n = 5)*‡ | (n = 5) | (n = 4)* | X (n = 5)*‡ | WKY*‡ | SHR | SHR + V | RLX*‡ | WKY | SHR | SHR + V | RLX |
| 250 | 98 ± 14 | 89 ± 15 | 68 ± 7 | 72 ± 10 | 1.04 ± 0.32 | 0.83 ± 0.12 | 0.86 ± 0.1 | 1.20 ± 0.18 | 30.6 ± 0.8 | 29.9 ± 1.6 | 29.1 ± 0.4 | 29.1 ± 0.3 |
| 200 | 92 ± 12 | 81 ± 13 | 59 ± 7 | 72 ± 13 | 0.93 ± 0.26 | 0.83 ± 0.13 | 0.82 ± 0.2 | 1.10 ± 0.19 | 30.6 ± 0.8 | 30.4 ± 2.1 | 29.4 ± 0.4 | 29.1 ± 0.2 |
| 180 | 89 ± 11 | 81 ± 17 | 55 ± 6 | 68 ± 15 | 0.88 ± 0.17 | 0.86 ± 0.12 | 0.84 ± 0.1 | 1.12 ± 0.21 | 30.5 ± 0.7 | 30.1 ± 1.5 | 29.1 ± 0.3 | 29.1 ± 0.3 |
| 160 | 86 ± 11 | 79 ± 14 | 57 ± 6 | 64 ± 11 | 0.94 ± 0.12 | 0.82 ± 0.09 | 0.84 ± 0.1 | 1.12 ± 0.23 | 30.5 ± 0.6 | 30.0 ± 1.5 | 28.8 ± 0.4 | 29.0 ± 0.2 |
| 140 | 80 ± 10 | 78 ± 10 | 56 ± 5 | 57 ± 10 | 0.91 ± 0.09 | 0.83 ± 0.12 | 0.85 ± 0.1 | 1.16 ± 0.18 | 30.9 ± 0.8 | 30.2 ± 2.2 | 29.0 ± 0.2 | 29.1 ± 0.1 |
| 120 | 76 ± 7 | 72 ± 6 | 56 ± 4 | 56 ± 9 | 0.91 ± 0.05 | 0.80 ± 0.11 | 0.81 ± 0.1 | 1.11 ± 0.25 | 30.6 ± 0.7 | 30.2 ± 1.6 | 29.2 ± 0.1 | 29.0 ± 0.2 |
| 100 | 66 ± 3 | 66 ± 5 | 53 ± 4 | 54 ± 8 | 0.89 ± 0.07 | 0.73 ± 0.09 | 0.81 ± 0.1 | 1.09 ± 0.19 | 29.9 ± 0.6 | 30.3 ± 2.5 | 29.4 ± 0.1 | 29.5 ± 0.8 |
| 90 | 63 ± 3 | 63 ± 3 | 52 ± 1.6 | 52 ± 7 | 0.83 ± 0.11 | 0.65 ± 0.09 | 0.80 ± 0.1 | 1.10 ± 0.22 | 29.2 ± 0.4 | 30.0 ± 2.3 | 29.5 ± 0.1 | 29.1 ± 0.3 |

For Table 3, similar findings were obtained in RA compared to LA (shown in Table 2). Rat hearts were perfused in a Langendorff apparatus and paced on the RA while mapping optical action potentials (AP) from the RA, field-of-view was 3×3 mm². AP durations (APD), conduction velocity (CV) and the rise-time of AP upstrokes were measured as a function of cycle length in ms. For WKY, SHR and SHR+RLX, n=5 heart per group, for SHR+V treatment n=4 hearts. *p<0.05 vs. SHR; ‡p<0.05 vs. WKY, SHR and SHR+V, (ANCOVA)

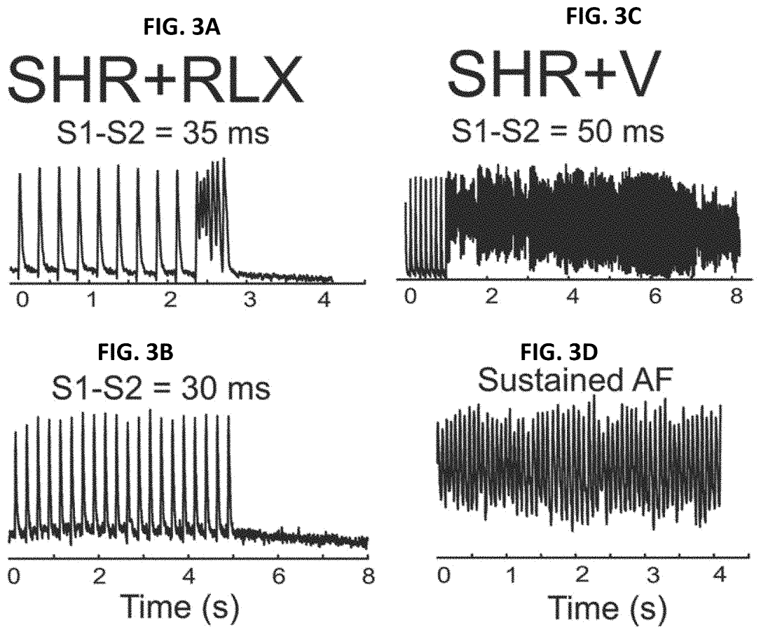

Effect of RLX on AF inducibility. A major and consistent finding was that RLX treatment of SHR for 2 weeks suppressed AF inducibility (n=7/8, one heart had an infarct) (FIG. 3A-B). In contrast, V treatment of SHR failed to suppress AF inducibility (n=4/4; p<0.01 vs. SHR+RLX) (FIGS. 3C and D). More robust attempts to elicit AF in RLX treated SHR hearts, such as varying the location of the pacing electrode and burst pacing (10 stimuli, 10 ms apart) on either the right or left atria, failed to elicit AF. In rare cases, the S2 impulse produced a non-sustained arrhythmia of <10 beats (FIG. 3A).

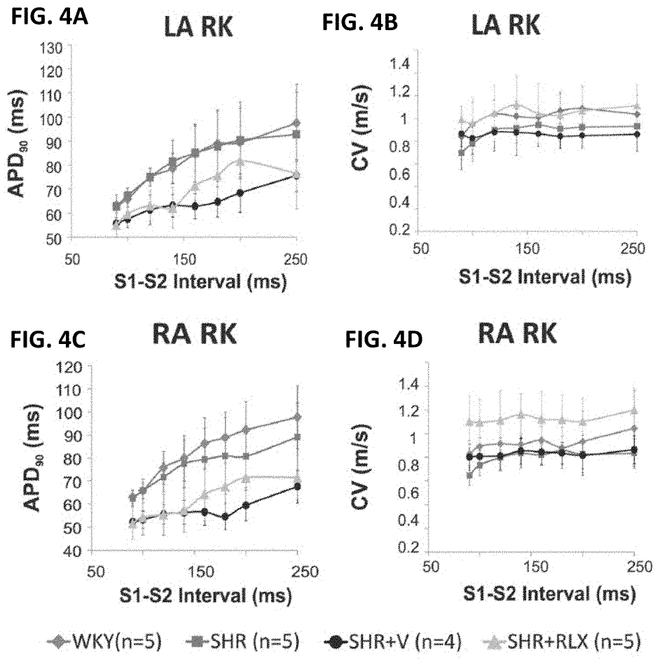

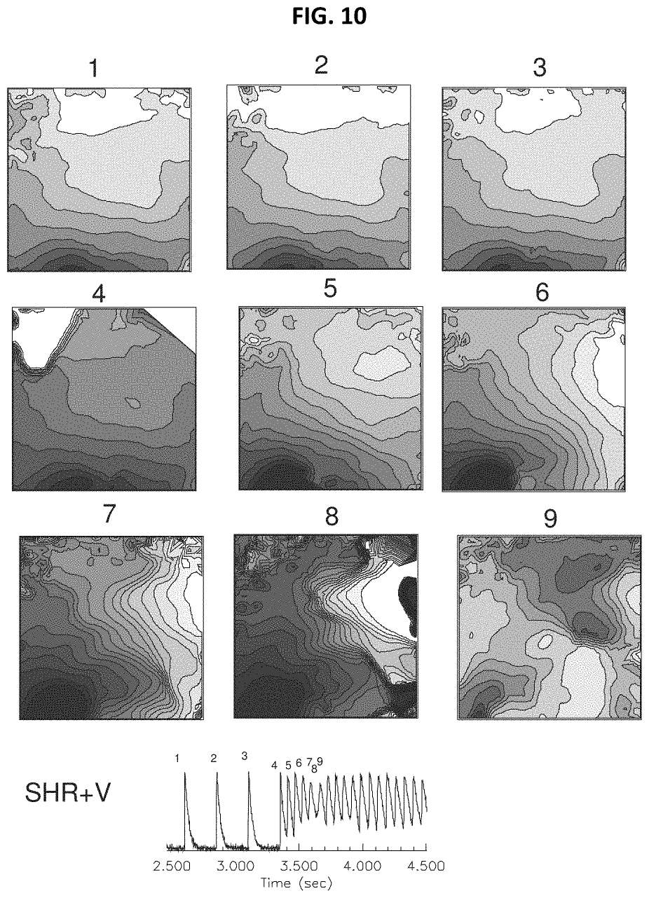

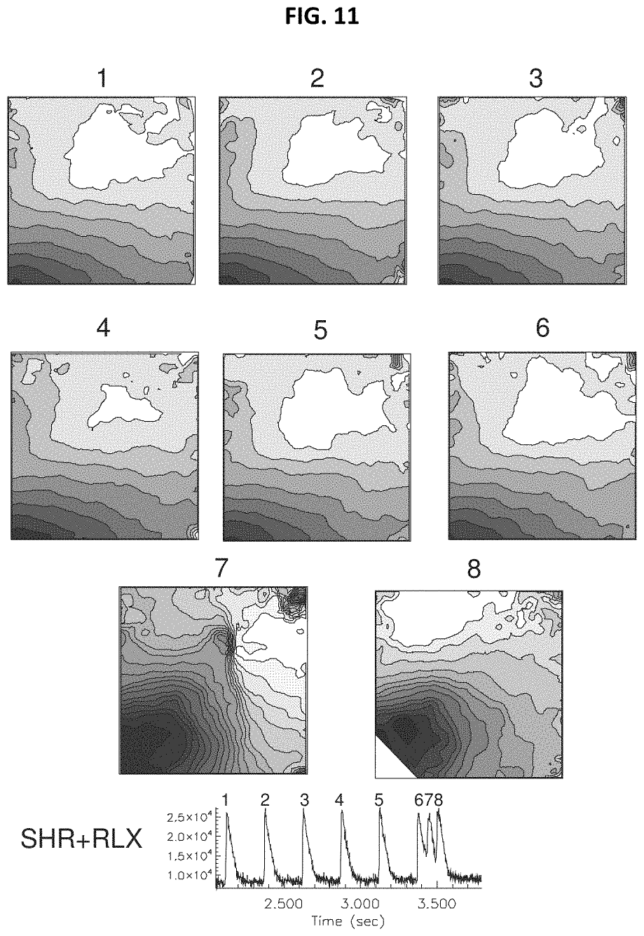

The mean RFs for SHR+V (51±4.3 ms, n=4) and SHR+RLX (5010 ms, n=5) left atria were not significantly different (p=NS). CV and APD restitution kinetics (RK) were measured from the RA and LA of WKY, SHR (untreated and treated with vehicle were combined) and SHR+RLX. FIG. 4 (right) shows a marked effect of RLX on the RK of CV of LA and RA compared to SHR hearts; namely a large increase in CV particularly for short S1-S2 intervals and a less-steep RK curve. RLX treatment did not significantly alter the slope of APD RK curves (left) for LA and RA. RLX treated SHR hearts had shorter APD₉₀ RK curves compared to SHR+V and WKY hearts consistent with APD₉₀ in Table 1. Activation maps of paced beats (S1), the premature beat (S2) and the first spontaneous beat are shown for an SHR+V and an SHR+RLX atrium (FIGS. 10A-B, respectively). The slower CV of the premature pulse and of the first spontaneous reentrant beat in SHR+V atria helps to sustain AF.

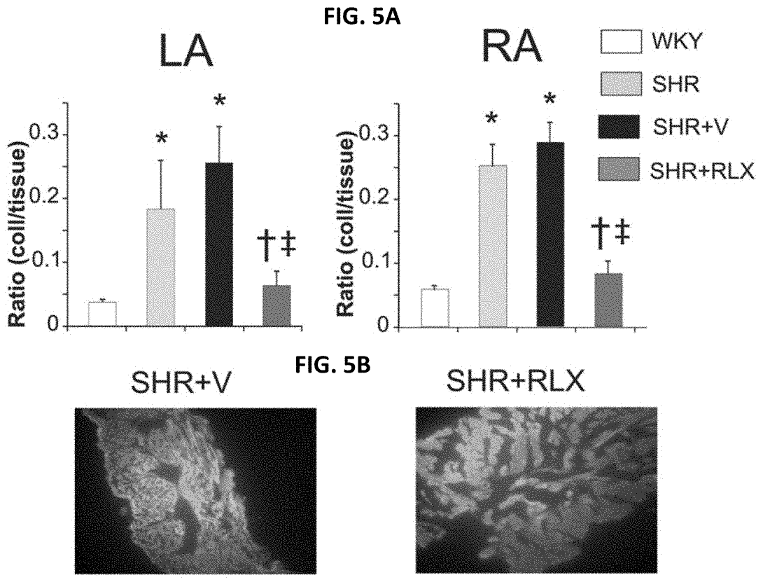

Histological findings. Differences in the level of fibrosis in the LA and RA of the different groups are shown in FIG. 5. SHR had a significantly greater collagen to tissue ratio in both the RA and LA compared to WKY (p<0.05). There was no significant difference in collagen to tissue ratio in both the RA and LA between SHR and SHR+V. However, RLX treatment attenuated the fibrosis within 2 weeks since SHR+RLX had a significantly lower collagen/tissue ratio when compared to SHR and SHR+V (p<0.05). SHR+V left atrial (LA) cardiomyocytes had a significant level of hypertrophy with greater cross-sectional area of LA myocytes (CSA=146.9±07.2 μm²) compared to WKY+V (95.5±10.6 m², p<0.01). The CSA of WKY+RLX atrial myocytes (96.9±3.3 μm²) did not differ from that of WKY+V. However, the CSA of LA cardiomyocytes from SHR+RLX was significantly less (100.8±2.98 m², p<0.05) than that of SHR+V and not significantly different from either WKY group. Thus, RLX appeared to reverse atrial myocyte hypertrophy in SHR hearts.

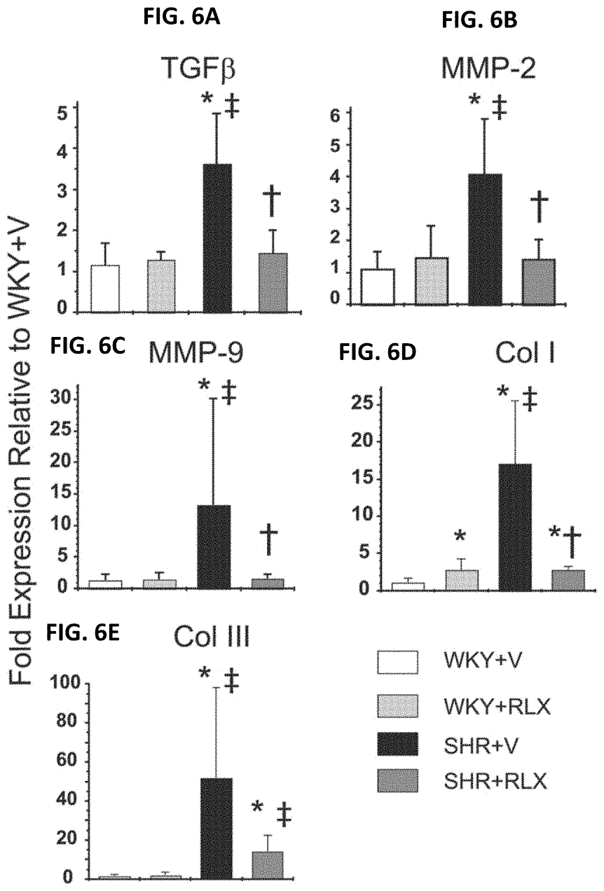

Effect of RLX on Cx 43 phosphorylation and Fibrosis-related Transcripts. The effect of RLX treatment on the relative phosphorylation of connexin 43 in SHR right atria was assessed by Western blot analysis, using the differential molecular weight of phosphorylated (43 kD) to non-phosphorylated connexin 43 (40 kD). Proteins from RLX-treated SHR showed a significantly greater ratio in the band intensity of the 43 to 40 kD proteins (SHR+RLX, 5.74±1.46; SHR+V, 2.15±1.26; n=4/group, p<0.01). The effect of RLX versus V-treatment on fibrosis related transcripts was examined by RT-PCR from RNA isolated from the left atria (LA) of 4-5 rats per group (WKY+V, WKY+RLX, SHR+V, SHR+RLX) (FIG. 6). TGFβ, MMP-2, MMP-9, collagen I, and collagen III transcripts were all significantly elevated in SHR+V versus WKY+V (p<0.05 or less). In WKY, RLX-treatment did not alter fibrosis-related transcripts (FIG. 6). In contrast, RLX-treatment significantly reduced all the transcripts except for collagen III, which exhibited a marked trend towards a decrease. For TGFβ, MMP-2, and MMP-9, transcripts levels in SHR+RLX were not different from their levels in WKY+V or WKY+RLX groups. Collagen I transcripts levels, while significantly reduced relative to SHR+V, remained somewhat elevated relative to WKY groups. Collagen III transcripts followed a similar pattern.

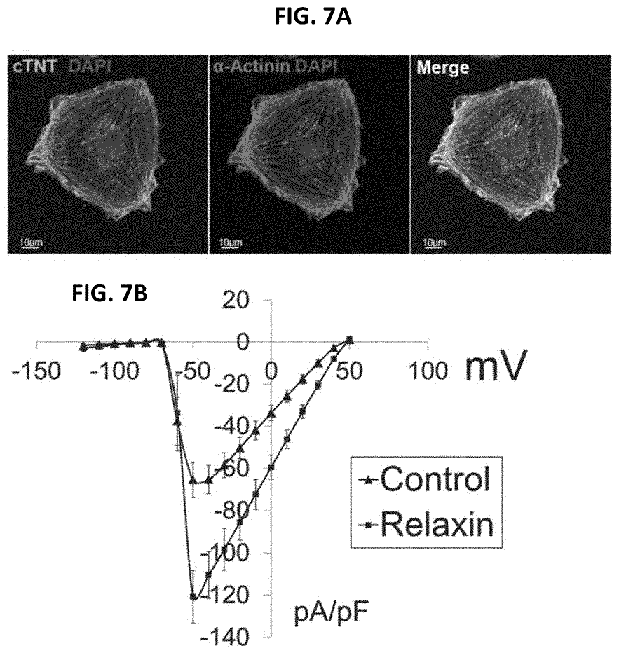

RLX upregulates I_{Na} in human iPS-CMs independent of fibrosis. A main electrophysiological change caused by RLX is a marked increase in CV which is difficult to attribute solely to reduced fibrosis and/or altered expression, localization and/or phosphorylation of connexin-43. Alternatively, large increases in CV are more readily caused by an increase in current density of voltage-gated sodium channels, I_{Na}. To test the effects of RLX on I_{Na} and the relevance of the findings in rat hearts to human hearts, the effects of RLX on I_{Na} density in human cardiomyocytes derived from inducible pluripotent stem cells (iPS-CMs) were tested. Human iPS-CMs were cultured with vehicle or 0.1 μM RLX for 48 hours then I_{Na} density was measured using the whole-cell voltage-clamp technique (see methods in supplement). Treatment of human iPS-CMs with RLX increased the peak I_{Na} density by ˜2-fold without altering the characteristics of the current-to-voltage relationship (FIG. 7). RLX did not alter I_{Na} acutely requiring at least 24 hours to upregulate the current. Human iPS-CMs largely represent mature human ventricular myocytes that exhibit low levels of inwardly rectifying K⁺ current. RLX (100 nM) was also found to upregulate I_{Na} density of guinea pig atrial myocytes in 24-72 hours. The time needed to enhance I_{Na} in cultured iPS-CMs is a strong indicator of a genomic upregulation of Nav1.5 that occurs independently of the anti-fibrotic effects of RLX and provides a compelling proof-of-concept that RLX may suppress AF in human hearts.

### DISCUSSION

The main findings are that SHR hearts have a higher susceptibility to AF triggered by a single premature impulse. SHR atria had a slower CV and higher levels of collagen deposition (i.e. fibrosis). RLX-treatment of SHR animals for 2 weeks significantly reversed fibrosis and hypertrophy, increased atrial CV, and suppressed AF.

Atrial Fibrosis and AF. Atrial fibrosis has been implicated in the pathogenesis of AF but a direct link between fibrosis and AF has not been established. Atrial tissue fibrosis is nevertheless a most consistent finding in patients and animal models of AF (Frustaci, et al., Chest. 1991; 100:303-306). The histological studies confirm that SHR hearts are fibrotic and hypertrophic compared to controls. In addition, SHR atria are characterized by conduction abnormalities that provide a basis for lines of conductional block that promote re-entry as seen in optical mapping studies. The major mechanisms that have been proposed for the initiation and maintenance of AF are the multiple wavelet theory (Moe, G K, Arch Int Pharmacodyn Ther. 1962; 140:183-188), focal activity hypothesis (Haissaguerre, et al., New Engl J Med. 1998; 339:659-666) and single circuit reentrant theory (Moe, G K, Rev Physiol Bioch P. 1975; 72:55-81). The optical mapping studies were consistent with AF generated by co-existing reentrant circuits with varying origins which supports the multiple-wavelet theory as the mechanism of AF.

Anti-fibrotic and anti-arrhythmic properties of RLX and its clinical relevance. RLX mediates effects on the cardiovascular system by activating a wide range of signaling pathways via the RLX family peptide receptor 1 (RXFP1), a G-protein coupled receptor that leads to an acute elevation of cyclic AMP (cAMP) and nitric oxide (NO) (Conrad, et al., Curr Hypertens Rep. 2011; 13:409-420; Du, et al., Nat Rev Cardiol. 2010; 7:48-58). In other studies, RLX has been shown to inhibit fibroblast proliferation, differentiation, collagen synthesis, collagen deposition and increase MMP-2 expression, which most likely contributed to an increase in collagen degradation and a decrease in collagen deposition (Samuel, et al., Endocrinology. 2004; 145:4125-4133). The results demonstrate the increased collagen I deposition, transcripts encoding the pro-fibrotic cytokine TGFβ, the major extracellular matrix fibrotic component collagen I, and MMP-2 and -9 in SHR atria relative to normotensive WKY, similar to previous reports for SHR LV and/or atrial tissues (Choisy, et al., Hypertension. 2007; 49:498-505; Conrad, et al., Circulation. 1995; 91:161-170). It was also observed a RLX-induced decrease in atrial collagen I and collagen transcripts, and TGFβ transcripts, similar to that reported for the SHR-LV and in a model of interstitial renal fibrosis (Lekgabe, et al., Hypertension. 2005; 46:412-418; Samuel, et al., Endocrinology. 2004; 145:4125-4133; Garber, et al., Kidney Int. 2001; 59:876-882) and consistent with a role for inhibition of TGFβ expression or signaling in the reversal of cardiac (and other organ) fibrosis by RLX (Heeg, et al., Kidney Int. 2005; 68:96-109). A decrease in RNA encoding MMP-2 and MMP-9 in response to RLX-treatment was observed, whereas an increase in MMP-2 activity has been previously observed in SHR ventricles treated with RLX (Samuel, et al., Endocrinology. 2004; 145:4125-4133). However, the observations are consistent with reports that in atria from rats or dogs subjected to interventions that increase AF susceptibility, fibrosis and MMP expression/activity were both elevated (Boixel, et al., Journal of the American College of Cardiology. 2003; 42:336-344; Moe, et al., J Card Fail. 2008; 14:768-776) and that MMP inhibition reversed atrial cardiomyocyte hypertrophy, MMP activity, collagen deposition, and AF-inducibility (Moe, et al., J Card Fail. 2008; 14:768-776). Targeting fibrosis has been attempted with ACE inhibitors, ARBs, and a novel compound Pirfenidone. However, most of these studies have examined models of heart failure, which is less commonly associated with AF than hypertension. Pirfenidone has been shown to reverse fibrosis and attenuate AF in a CHF canine model (Lee, et al., Circulation. 2006; 114:1703-1712). Pirfenidone treatment achieved reversal of atrial fibrosis and reduced vulnerability of AF after burst pacing but did not generate a significantly greater increase in atrial CV. In contrast the data shows that treatment with RLX reduces AF inducibility, reverses atrial fibrosis and hypertrophy, increases CV and decreases action potential duration (APD).

It is important to note that RLX treatment of SHRs for 1-week was ineffective at suppressing AF and that longer RLX-treatment was necessary because remodeling of the ECM and/or gap-junctions may be reversed, albeit slowly. Reversal of fibrosis is a slow process due to the slow collagen turnover rate of 5% per day in healthy hearts (Weber, et al., Ann NY Acad Sci. 1995; 752:286-299). Enhanced atrial fibrosis can in turn alter connexin-43 expression and its redistribution to lateral cell borders, creating a barrier to impulse propagation by reducing inter-myocyte coupling and CV (Burstein, et al., J Am Coll Cardiol. 2008; 51:802-809). However, it is difficult to evaluate the amount of connexin disruption that is required to produce a significant change of CV and an alternative mechanism is to increase CV by an upregulation of I_{Na} density. The pleiotropic effects of RLX and its relevance to human hearts was demonstrated by testing its effects on cultured human iPS-CMs. Independent of fibrosis, RLX increased sodium current density in 48 hours indicating that RLX acted at fibroblasts to remodel ECM and human myocytes to alter ion channel expression.

The actions of 2-weeks of RLX-treatment differ from the acute effects of RLX. In rat hearts, RLX was found to bind to atrial tissue (Osheroff, et al., Proc Natl Acad Sci USA. 1992; 89:2384-2388), increase heart rate (Ward, et al., Biochem Biophys Res Commun. 1992; 186:999-1005), prolong APD by inhibiting the I_{t,o} K⁺ current (Piedras-Renteria, et al., Am J Physiol. 1997; 272:H1791-1797) and increase Ca²⁺ influx due to APD prolongation (Piedras-Renteria, et al., Am J Physiol. 1997; 272:H1798-1803). The acute effect of RLX on heart rate was readily measured in perfused hearts but the longer-term effects of RLX on increased atrial CV and reduced APD₉₀ relative to SHR+V controls imply additional direct effects on ion channel properties and/or expression as well as its anti-fibrotic effects.

Efficacy and Safety. RLX has been under clinical trials for acute heart failure with a completed 234-patient phase 2 and an ongoing 160-patient phase 3 (Ponikowski, et al., Am Heart J. 2012; 163:149-155 e141). Reports have confirmed the safety of RLX-infusion in humans (up to 0.96 mg/kg)/day) and have noted a vasodilatory effect in patients with HF, but RLX therapy did not always improve renal functions (Voors, et al., Eur J Heart Fail. 2011; 13:961-967). The clinical trials to date have addressed potential benefits of short-term treatment in vasodilation, but have not examined whether other pathways mediated by RLX can be exploited to provide long-term therapeutic benefits.

RLX has the anticipated anti-fibrotic effects on the atria but reversal of fibrosis may not be sufficient to explain the marked increase in CV which is the predominant mechanism for AF suppression. As proof-of-concept that RLX modulates cardiac properties independent of fibrosis and is relevant to human AF, the effects of RLX on the voltage-gated sodium current in cultured human iPS-CMs was tested. RLX-treatment for 48 hours markedly upregulated I_{Na} density (from −22.95±5.8 to −38.64±10 pA/pF, mean±SEM) most likely by a genomic mechanism which could explain the increase in CV and faster AP rise-time. Hence, longer-term treatment with RLX suppresses AF in part by reversing fibrosis, enhancing connexin-43 phosphorylation and upregulating voltage-gated Na⁺ channels.

### Example 2

### Treatment of Atrial Fibrillation in a Human Subject

This example describes a particular method that can be used to treat atrial fibrillation in a human subject by administration of one or more RLX polypeptides including an amino acid sequence of mature RLX, or a precursor of mature RLX, wherein the amino acid sequence has up to four amino acid substitutions, wherein the polypeptide specifically binds the RLX receptor; or a nucleic acid molecule encoding the polypeptide, to treat a subject with atrial fibrillation. Although particular methods, dosages, and modes of administrations are provided, one skilled in the art will appreciate that variations can be made without substantially affecting the treatment.

Based upon the teaching disclosed herein, atrial fibrillation, such as first detected, paroxysmal, persistent or chronic atrial fibrillation, can be treated by administering a therapeutically effective amount of a RLX polypeptide thereby inhibiting atrial fibrillation.

Briefly, the method can include screening subjects (for example, using electrocardiogram to determine if they have atrial fibrillation, such as first detected, paroxysmal, persistent or chronic atrial fibrillation). Subjects having atrial fibrillation are selected. In one example, a clinical trial would include half of the subjects following an established protocol for treatment of atrial fibrillation (such as a beta blocker). The other half would follow the established protocol for treatment of atrial fibrillation (such as treatment with a beta blocker) in combination with administration of the agents including RLX polypeptides or a nucleic acid molecule encoding the RLX polypeptide (as described above). In another example, a clinical trial would include half of the subjects following the established protocol for treatment of atrial fibrillation (such as a beta blocker). The other half would receive one or more RLX polypeptides or a nucleic acid molecule encoding the polypeptide (as described above).

### Screening Subjects

In particular examples, the subject is first screened to determine if they have atrial fibrillation. Examples of methods that can be used to screen for atrial fibrillation include a combination of measuring electrical conduction through heart of a subject using an electrocardiogram. Detection of the sinus rhythm of the electrical conduction of the heart allows detection of an atrial fibrillation. The detection of cardiac arrhythmia by electrocardiogram is indicative that the subject has atrial fibrillation and is a candidate for receiving the therapeutic compositions disclosed herein.

Pre-screening is not required prior to administration of the therapeutic compositions disclosed herein.

### Pre-Treatment of Subjects

In particular examples, the subject is treated prior to administration of a therapeutic agent that includes one or more of the disclosed RLX polypeptides or a nucleic acid molecule encoding the polypeptide. However, such pre-treatment is not always required, and can be determined by a skilled clinician. For example, the subject can be treated with an established protocol for treatment of atrial fibrillation.

### Administration of Therapeutic Compositions

Following subject selection, a therapeutic effective dose of the RLX polypeptides or a nucleic acid molecule encoding the polypeptide is administered to the subject (such as an adult human either at risk for atrial fibrillation or known to have atrial fibrillation). The methods can include administering an RLX polypeptide including the amino acid sequence of mature RLX, a precursor of mature RLX, or a nucleic acid molecule encoding the polypeptide. Additional agents, such as anti-atrial fibrillation agents, can also be administered to the subject simultaneously or prior to or following administration of the disclosed agents. Administration can be achieved by any method known in the art, such as oral administration, inhalation, intravenous, intramuscular, intraperitoneal, subcutaneous, transcutaneous, or direct injection into tissue such as the myocardium, pericardium or pericardial space.

The amount of the composition administered to prevent, reduce, inhibit, and/or treat atrial fibrillation or a condition associated with it depends on the subject being treated, the severity of the disorder, and the manner of administration of the therapeutic composition. Ideally, a therapeutically effective amount of an agent is the amount sufficient to prevent, reduce, and/or inhibit, and/or treat the condition (e.g., atrial fibrillation) in a subject without causing a substantial cytotoxic effect in the subject. An effective amount can be readily determined by one skilled in the art, for example using routine trials establishing dose response curves. In addition, particular exemplary dosages are provided above. The therapeutic compositions can be administered in a single dose delivery, via continuous delivery over an extended time period, in a repeated administration protocol (for example, by a daily, weekly, or monthly repeated administration protocol). In one example, therapeutic agents that include one or more RLX polypeptides or a nucleic acid molecule encoding the polypeptide are administered intravenously to a human. As such, these compositions may be formulated with an inert diluent or with a pharmaceutically acceptable carrier.

Administration of the therapeutic compositions can be taken long term (for example over a period of months or years).

### Assessment

Following the administration of one or more therapies, subjects having atrial fibrillation (for example, persistent atrial fibrillation) can be monitored for reductions in atrial fibrillation, or reductions in one or more clinical symptoms associated with atrial fibrillation. In particular examples, subjects are analyzed one or more times, starting 7 days following treatment. Subjects can be monitored using any method known in the art. For example, the electrical conduction through the heart of the subject can be measured using an electrocardiogram.

It will be apparent that the precise details of the methods or compositions described may be varied or modified without departing from the spirit of the described embodiments. We claim all such modifications and variations that fall within the scope and spirit of the claims below.

1. A method of delaying progression of atrial fibrillation in a subject, comprising:
selecting a subject with persistent atrial fibrillation;
administering a therapeutically effective amount of a controlled release formulation comprising relaxin to a pericardial space of the subject;
wherein administering the therapeutically effective amount of the controlled release formulation comprising relaxin to the pericardial space of the subject delays progression from persistent atrial fibrillation to chronic atrial fibrillation in the subject.

2. A method of inhibiting or treating atrial fibrillation in a subject, comprising:
administering systemically a therapeutically effective amount of relaxin to a subject with or at risk of atrial fibrillation, thereby inhibiting or treating atrial fibrillation in the subject.

3. The method of claim 2, wherein the relaxin is relaxin-1, relaxin-2, or relaxin-3.

4. The method of claim 2, wherein the subject has one of first detected, paroxysmal, persistent, or chronic atrial fibrillation.

5. The method of claim 2, wherein administering a therapeutically effective amount of relaxin to the subject comprises administering from 0.05 to 0.5 mg/kg/day relaxin to the subject.

6. The method of claim 5, wherein administering a therapeutically effective amount of relaxin to the subject comprises administering about 0.5 mg/kg/day relaxin to the subject.

7. The method of claim 2, wherein administering a therapeutically effective amount of relaxin to the subject comprises administering the relaxin to the subject for at least seven days.

8. The method of claim 7, wherein administering a therapeutically effective amount of relaxin to the subject comprises administering the relaxin to the subject for seven days.

9. The method of claim 2, wherein administering a therapeutically effective amount of relaxin to the subject comprises administering the relaxin to the subject for at least two weeks.

10. The method of claim 2, wherein administering a therapeutically effective amount of relaxin to the subject comprises administering the relaxin to the subject for no more than two weeks.

11. The method of claim 2, further comprising administering an anti-fibrotic agent to the subject.

12. The method of claim 11, wherein the anti-fibrotic agent is an angiotensin converting enzyme (ACE) inhibitor, an angiotensin receptor blocker or a TGF-β inhibitor.

13. The method of claim 2, wherein administering the therapeutically effective amount of relaxin to the subject increases cardiac sodium channel current in the subject.

14. The method of claim 2, wherein treating the subject comprises an increase in Nav1.5 expression in cardiac tissue.

15. The method of claim 2, further comprising selecting the subject with or at risk of atrial fibrillation.

16. The method of claim 2, wherein administering systemically the therapeutically effective amount of relaxin comprises intravenous administration of the relaxin.

17. The method of claim 2, wherein the subject is a human subject.

18. The method of claim 2, wherein administering systemically comprises subcutaneous administration.

19. The method of claim 2, wherein administering systemically the therapeutically effective amount of relaxin comprises:
a first treatment comprising systemic administration to the subject of from 0.01 mg/kg/day to 0.1 mg/kg/day relaxin for two weeks using a continuous release pump;
a second treatment comprising systemic administration to the subject of from 0.01 mg/kg/day to 0.1 mg/kg/day relaxin for two weeks using a continuous release pump; and
wherein the second treatment is administered about 6 months, about 7 months, or about 8 months following administration of the first treatment, thereby inhibiting or treating atrial fibrillation in the subject.

20. A method of treating atrial fibrosis in a subject, comprising:
administering systemically a therapeutically effective amount of relaxin to a subject with or at risk of atrial fibrosis, thereby inhibiting or treating atrial fibrosis in the subject.


SEQUENCE LISTING (16 sequences)

SEQ ID NO: 1 - protein, 24 aa, homo sapiens
RPYVALFEKCCLIGCTKRSLAKYC

SEQ ID NO: 2 - protein, 28 aa, homo sapiens
KWKDDVIKLCGRELVRAQIAICGMSTWS

SEQ ID NO: 3 - protein, 24 aa, homo sapiens
QLYSALANKCCHVGCTKRSLARFC

SEQ ID NO: 4 - protein, 29 aa, homo sapiens
DSWMEEVIKLCGRELVRAQIAICGMSTWS

SEQ ID NO: 5 - DNA, 20 bp, artificial sequence
  DNA primer
gcaccaccgaggattatgac

SEQ ID NO: 6 - DNA, 20 bp, artificial sequence
  DNA primer
cacccacagtggacatagca

SEQ ID NO: 7 - DNA, 22 bp, artificial sequence
  DNA primer
cctctgcatgaagacgacataa

SEQ ID NO: 8 - DNA, 20 bp, artificial sequence
  DNa primer
ggtcaggtttagagccacga

SEQ ID NO: 9 - DNA, 21 bp, artificial sequence
  DNA primer
catgttcagctttgtggacct

SEQ ID NO: 10 - DNA, 20 bp, artificial sequence
  DNA primer
gcagctgacttcagggatgt

SEQ ID NO: 11 - DNA, 20 bp, artificial sequence
  DNa primer
tcccctggaatctgtgaatc

SEQ ID NO: 12 - DNA, 20 bp, artificial sequence
  DNA primer
tgagtcgaattggggagaat

SEQ ID NO: 13 - DNA, 19 bp, artificial sequence
  DNA primer
cctggaaagggctcaacac

SEQ ID NO: 14 - DNA, 22 bp, artificial sequence
  DNA primer
cagttcttctctgtggagctga

SEQ ID NO: 15 - DNA, 19 bp, artificial sequence
  DNA primer
agctggtcatcaatgggaa

SEQ ID NO: 16 - DNA, 19 bp, artificial sequence
  DNA primer
atttgatgttagcgggatc